(19) United States
(12) Patent Application Publication
(10) Pub. No.: US 2024/0190844 A1
(43) Pub. Date: Jun. 13, 2024

(54) PYRIDAZINONES AS PARP7 INHIBITORS

(71) Applicant: Ribon Therapeutics, Inc., Cambridge, MA (US)

(72) Inventors: Nicholas Robert Perl, Somerville, MA (US); Melissa Marie Vasbinder, Newton, MA (US); Laurie B. Schenkel, Somerville, MA (US); Kevin Wayne Kuntz, Woburn, MA (US)

(21) Appl. No.: 17/772,679

(22) PCT Filed: Oct. 29, 2020
(86) PCT No.: PCT/US20/57819

§ 371 Date: Apr. 28, 2022

Related U.S. Application Data
(60) Provisional application No. 62/927,929, filed on Oct. 30, 2019.

(51) Int. Cl. C07D 403/12; A61K 31/501; A61K 31/506; C07D 401/12; C07D 403/14; C07D 417/12

(52) U.S. Cl. CPC C07D 403/12; A61K 31/501; A61K 31/506; C07D 401/12; C07D 403/14; C07D 417/12

(57) ABSTRACT

The present invention relates to pyridazinones and related compounds which are inhibitors of PARP7 and are useful in the treatment of cancer.

### FIELD OF THE INVENTION

The present invention relates to pyridazinones and related compounds which are inhibitors of PARP7 and are useful in the treatment of cancer.

### BACKGROUND OF THE INVENTION

Poly(ADP-ribose) polymerases (PARPs) are members of a family of seventeen enzymes that regulate fundamental cellular processes including gene expression, protein degradation, and multiple cellular stress responses (M. S. Cohen, P. Chang, Insights into the biogenesis, function, and regulation of ADP-ribosylation. Nat Chem Biol 14, 236-243 (2018)). The ability of cancer cells to survive under stress is a fundamental cancer mechanism and an emerging approach for novel therapeutics. One member of the PARP family, PARP1, has already been shown to be an effective cancer target in connection to cellular stress induced by DNA damage, either induced by genetic mutation or with cytotoxic chemotherapy, with four approved drugs in the clinic and several others in late stage development (A. Ohmoto, S. Yachida, Current status of poly(ADP-ribose) polymerase inhibitors and future directions. Onco Targets Ther 10, 5195-5208 (2017)).

The seventeen members of the PARP family were identified in the human genome based on the homology within their catalytic domains (S. Vyas, M. Chesarone-Cataldo, T. Todorova, Y. H. Huang, P. Chang, A systematic analysis of the PARP protein family identifies new functions critical for cell physiology.Nat Commun 4, 2240) (2013)). However, their catalytic activities fall into 3 different categories (S. Vyas et al., Family-wide analysis of poly (ADP-ribose) polymerase activity. Nat Commun 5, 4426 (2014)). The majority of PARP family members catalyze the transfer of mono- ADP-ribose units onto their substrates (monoPARPs), while others (PARP1, PARP2, TNKS, TNKS2) catalyze the transfer of poly-ADP-ribose units onto substrates (polyPARPs). Finally, PARP13 is thus far the only PARP for which catalytic activity could not be demonstrated either in vitro or in vivo.

The aryl hydrocarbon receptor (AHR) is a ligand-activated transcription factor involved in regulating multiple cellular functions including proinflammatory responses and xenobiotic metabolism (S. Feng. Z. Cao, X. Wang, Role of aryl hydrocarbon receptor in cancer. Biochim Biophys Acta 1836, 197-210 (2013); and B. Stockinger, P. Di Meglio, M. Gialitakis, J. H. Duarte, The aryl hydrocarbon receptor: multitasking in the immune system. Annu Rev Immunol 32, 403-432 (2014)). The AHR can be activated by a broad number of ligands including endogenous tryptophan metabolites such as kynurenine (C. A. Opitz et al., An endogenous tumour-promoting ligand of the human aryl hydrocarbon receptor. Nature 478, 197-203 (2011)) and certain polycyclic aromatic hydrocarbons such as 2,3,7,8-tetrachlorodibenzo-p-dioxin (TCDD) (K. W. Bock, Toward elucidation of dioxin-mediated chloracne and Ah receptor functions. Biochem Pharmacol 112, 1-5 (2016)). Activation of the AHR induces target gene expression including genes involved in metabolism such as cytochrome P4501A1 and P4501B1. Activation of AHR also leads to an increase in the AHR target gene, TCDD-inducible poly(ADP-ribose)polymerase (TIPARP, also referred to as PARP7), which functions as a negative regulator of certain AHR transcriptional targets (L. MacPherson et al., Aryl hydrocarbon receptor repressor and TIPARP (ARTD14) use similar, but also distinct mechanisms to repress aryl hydrocarbon receptor signaling. Int J Mol Sci 15, 7939-7957 (2014); and L. MacPherson et al., 2,3,7,8-Tetrachlorodibenzo-p-dioxin poly(ADP-ribose) polymerase (TIPARP, ARTD14) is a mono-ADP-ribosyltransferase and repressor of aryl hydrocarbon receptor transactivation. Nucleic Acids Res 41, 1604-1621 (2013)).

PARP7 can also be regulated by other transcription factors and signaling pathways including androgen receptor (E. C. Bolton et al., Cell- and gene-specific regulation of primary target genes by the androgen receptor. Genes Dev 21, 2005-2017 (2007)), platelet derived growth factor (J. Schmahl, C. S. Raymond, P. Soriano, PDGF signaling specificity is mediated through multiple immediate early genes. Nat Genet 39, 52-60 (2007)) and hypoxia inducible factor 1 (N. Hao et al., Xenobiotics and loss of cell adhesion drive distinct transcriptional outcomes by aryl hydrocarbon receptor signaling. Mol Pharmacol 82, 1082-1093 (2012)). The PARP7 gene is located on chromosome 3 (3q25) in a region that is frequently amplified in cancers of squamous histology (http://www.cbioportal.org/index.do?session_id=5ae1bcde498eb8b3d565d8b2). A genome-wide association study identified 3q25 as susceptibility loci for ovarian cancer suggesting a role for PARP7 in this cancer type (E. L. Goode et al., A genome-wide association study identifies susceptibility loci for ovarian cancer at 2q31 and 8924. Nat Genet 42, 874-879 (2010)). PARP7 has multiple cellular functions. In the context of AHR signaling PARP7 acts as a negative feedback mechanism to regulate the expression of P4501A1 and P4501B1 (L. MacPherson et al., Aryl hydrocarbon receptor repressor and TIPARP (ARTD14) use similar, but also distinct mechanisms to repress aryl hydrocarbon receptor signaling. Int J Mol Sci 15, 7939-7957 (2014). and L. MacPherson et al., 2,3,7,8-Tetrachlorodibenzo-p-dioxin poly(ADP-ribose) polymerase (TIPARP, ARTD14) is a mono-ADP-ribosyltransferase and repressor of aryl hydrocarbon receptor transactivation. Nucleic Acids Res 41, 1604-1621 (2013)). PARP7 has also been described to ADP-ribosylate liver X receptors which leads to the modulation of their transcriptional activity (C. Bindesboll et al., TCDD-inducible poly-ADP-ribose polymerase (TIPARP/PARP7) mono-ADP-ribosylates and co-activates liver X receptors. Biochem J 473, 899-910 (2016). During viral infection PARP7 can bind to Sindbis virus (SINV) to promote viral RNA degradation (T. Kozaki et al., Mitochondrial damage elicits a TCDD-inducible poly(ADP-ribose) polymerase-mediated antiviral response. Proc Natl Acad Sci U S A 114, 2681-2686 (2017)). Also in the context of viral infection, AHR-induced PARP7 can interact with TBK1, a major kinase that is activated during the onset of pathogen-associated molecular pattern pathways leading to an activation of the Type I interferon response and antiviral immunity (T. Yamada et al., Constitutive aryl hydrocarbon receptor signaling constrains Type I interferon-mediated antiviral innate defense. Nat Immunol 17, 687-694 (2016)). PARP7 was shown to ADP-ribosylate TBK1 which prevents its activation, thereby repressing the Type I interferon response.

Based on these results from viral infection one could hypothesize that cancer cells can use aberrantly expressed and/or activated PARP7 as a mechanism to evade the host immune system through suppression of the Type I interferons and thereby T cell mediated antitumor immunity. Indeed, in a recent genetic screen to identify tumor factors that suppress T cell activation PARP7 was identified as a hit (D. Pan et al., A major chromatin regulator determines resistance of tumor cells to T cell-mediated killing. Science 359, 770-775 (2018)). PARP7 knockout in a mouse melanoma cell line was shown to increase the proliferation and activation of co-cultured T cells suggesting that PARP7 inhibition may be a viable strategy to activate T cell mediated tumor killing. Thus, there is an ongoing need for the development of PARP7 inhibitors.

### SUMMARY OF THE INVENTION

The present invention is directed to a compound of Formula I:

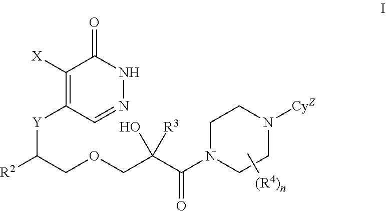

or a pharmaceutically acceptable salt thereof, wherein constituent members are defined below.

The present invention is further directed to a pharmaceutical composition comprising a compound of Formula I, or a pharmaceutically acceptable salt thereof, and at least one pharmaceutically acceptable carrier.

The present invention is further directed to a method of inhibiting the activity of PARP7 comprising contacting a compound of Formula I, or a pharmaceutically acceptable salt thereof, with PARP7.

The present invention is further directed to a method of treating a disease or disorder in a patient in need of treatment, where the disease or disorder is characterized by overexpression or increased activity of PARP7, comprising administering to the patient a therapeutically effective amount of a compound of Formula I, or a pharmaceutically acceptable salt thereof.

The present invention is further directed to a method of treating cancer in a subject in need thereof, the method comprising administering to the subject a therapeutically effective amount of an agent that inhibits PARP7 activity, such as a compound of Formula I, or a pharmaceutically acceptable salt thereof. The present disclosure also provides uses of the compounds described herein in the manufacture of a medicament for use in therapy. The present disclosure also provides the compounds described herein for use in therapy.

### DETAILED DESCRIPTION

The present invention is directed to a compound of Formula I:

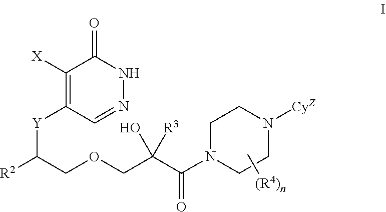

or a pharmaceutically acceptable salt thereof, wherein:

X is halo or CF₃;

Y is NR¹ or O;

R¹ is H or C₁-6 alkyl;

R² is H or C₁₋₆ alkyl; wherein said C₁₋₆ alkyl is optionally substituted with OR^{a};

or R¹ and R² together with the atoms to which they are attached form a 4-10 membered heterocycloalkyl group; wherein the 4-10 membered heterocycloalkyl group has at least one ring-forming carbon atom and 1, 2, or 3 ring-forming heteroatoms independently selected from N, O, and S; wherein a ring-forming carbon atom of the 4-10 membered heterocycloalkyl group is optionally substituted by oxo to form a carbonyl group; and wherein the 4-10 membered heterocycloalkyl group is optionally substituted with 1, 2, 3, or 4 substituents each independently selected from halo, D, OH, C₁₋₆ alkyl, C₁₋₆ haloalkyl, C₁₋₆ alkoxy, C₁₋₆ alkylamino, and C₁₋₆ dialkylamino;

R³ is H or C₁₋₆ alkyl;

each R⁴ is independently selected from D, C₁₋₆ alkyl, C₁₋₆ haloalkyl, and C₁₋₆ alkoxy;

Cy^{Z} is a 5-10 membered heteroaryl group substituted with R^{Z};

R^{Z} is halo, CN, C₁₋₆ alkyl, or C₁₋₃ haloalkyl;

R^{a} is selected from H, C₁₋₆ alkyl, and C₁₋₆ haloalkyl; and

n is an integer from 0 to 8.

In some embodiments, X is CF₃.

In some embodiments, X is halo. In some embodiments, X is Br.

In some embodiments, X is Br or CF₃.

In some embodiments, Y is NR¹. In some embodiments, Y is NH.

In some embodiments, Y is O.

In some embodiments, Y is NH or O.

In some embodiments, R¹ is H. In some embodiments, R¹ is C₁₋₆ alkyl.

In some embodiments, R² is H or C₁₋₃ alkyl, wherein said C₁₋₃ alkyl is optionally substituted with OR^{a}. In some embodiments, R² is H, methyl, or ethyl; wherein said methyl and ethyl are each optionally substituted with methoxy or trifluoromethoxy. In some embodiments, R² is H, methyl, ethyl, methoxymethyl, or trifluoromethoxymethyl.

In some embodiments, R² is C₁₋₆ alkyl optionally substituted with OR^{a}. In some embodiments, R² is C₁₋₆ alkyl optionally substituted with methoxy or trifluoromethoxy. In some embodiments, R² is methyl optionally substituted with methoxy or trifluoromethoxy. In some embodiments, R² is methyl, methoxymethyl, or trifluoromethoxymethyl. In some embodiments, R² is methyl.

In some embodiments, R¹ and R² together with the atoms to which they are attached form a 4-10 membered heterocycloalkyl group; wherein the 4-10 membered heterocycloalkyl group is optionally substituted with 1 or 2 substituents each independently selected from halo, D, OH,
C₁₋₆ alkyl, C₁₋₆ haloalkyl, C₁₋₆ alkoxy, and C₁₋₆ alkylamino. In some embodiments, R¹ and R² together with the atoms to which they are attached form an azetidine ring or isoindoline ring; each of which is optionally substituted with 1 or 2 substituents each independently selected from halo, D, OH, C₁₋₆ alkyl, C₁₋₆ haloalkyl, C₁₋₆ alkoxy, and C₁₋₆ alkylamino. In some embodiments, R¹ and R² together with the atoms to which they are attached form an azetidine ring optionally substituted with 1 or 2 substituents each independently selected from halo, D, OH, C₁₋₆ alkyl, C₁₋₆ haloalkyl, C₁₋₆ alkoxy, and C₁₋₆ alkylamino. In some embodiments, R¹ and R² together with the atoms to which they are attached form an isoindoline ring optionally substituted with 1 or 2 substituents each independently selected from halo, D, OH, C₁₋₆ alkyl, C₁₋₆ haloalkyl, C₁₋₆ alkoxy, and C₁₋₆ alkylamino.

In some embodiments, R¹ and R² together with the atoms to which they are attached form a 4-6 membered heterocycloalkyl group. In some embodiments, R¹ and R² together with the atoms to which they are attached form an azetidine ring or isoindoline ring. In some embodiments, R¹ and R² together with the atoms to which they are attached form an azetidine ring. In some embodiments, R¹ and R² together with the atoms to which they are attached form an isoindoline ring.

In some embodiments, R³ is H or C₁₋₆ alkyl. In some embodiments, R³ is H. In some embodiments, R³ is methyl. In some embodiments, R³ is H or methyl.

In some embodiments, each R⁴ is independently selected from halo and D. In some embodiments, each R⁴ is D.

In some embodiments, n is 0. In some embodiments, n is 8.

In some embodiments, n is 8; and each R⁴ is D.

In some embodiments, Cy^{Z} is a 5-6 membered heteroaryl group substituted with R^{Z}. In some embodiments, Cy^{Z} is a 5 membered heteroaryl group substituted with R^{Z}. In some embodiments, Cy^{Z} is a 6 membered heteroaryl group substituted with R^{Z}.

In some embodiments, Cy^{Z} is a pyrimidinyl, pyrazinyl, pyridinyl, or thiazolyl group substituted with R^{Z}. In some embodiments, Cy^{Z} is a pyrimidinyl, pyrazinyl, pyridinyl, or thiazolyl group substituted with R^{Z}. In some embodiments, Cy^{Z} is a pyrimidinyl, pyrazinyl, or pyridinyl group substituted with R^{Z}. In some embodiments, Cy^{Z} is a pyrimidinyl or pyrazinyl group substituted with R^{Z}. In some embodiments, Cy^{Z} is a thiazolyl group substituted with R^{Z}. In some embodiments, Cy^{Z} is a thiazolyl group substituted with R^{Z}.

In some embodiments, Cy^{Z} is selected from 5-(trifluoromethyl)pyrimidin-2-yl, 5-(trifluoromethyl)thiazol-2-yl, 5-(trifluoromethyl)pyrazin-2-yl, 5-cyanopyridin-2-yl, 5-(difluoromethyl)pyrimidin-2-yl, and 5-chloropyrimidin-2-yl.

In some embodiments, R^{Z} is halo, CN or C₁₋₃ haloalkyl. In some embodiments, R^{Z} is CN. In some embodiments, R^{Z} is C₁₋₃ haloalkyl.

In some embodiments, R^{Z} is selected from CF₃, CN, CF₂H, and Cl. In some embodiments, R^{Z} is selected from CF₃ and CF₂H. In some embodiments, R^{Z} is CF₃. In some embodiments, R^{Z} is CF₂H. In some embodiments, R^{Z} is CN. In some embodiments, R^{Z} is Cl.

In some embodiments, R^{a} is selected from C₁₋₆ alkyl and C₁₋₆ haloalkyl.

In some embodiments, R^{a} is selected from methyl and trifluoromethyl.

In some embodiments, the compound of Formula I has Formula II:

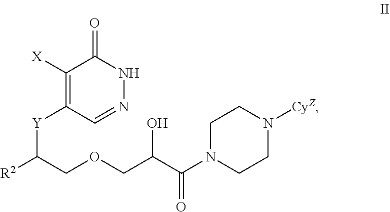

or a pharmaceutically acceptable salt thereof.

In some embodiments, the compound of Formula I has Formula IIIa:

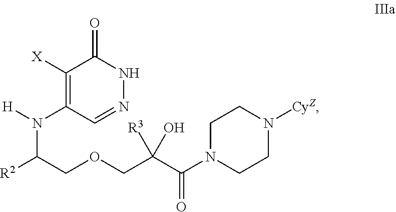

or a pharmaceutically acceptable salt thereof.

In some embodiments, the compound of Formula I has Formula IIIb:

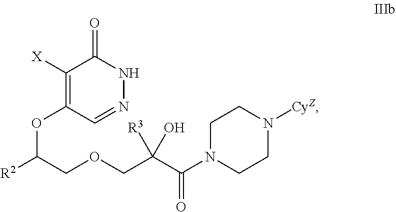

or a pharmaceutically acceptable salt thereof.

In some embodiments, the compound of Formula I has Formula IV:

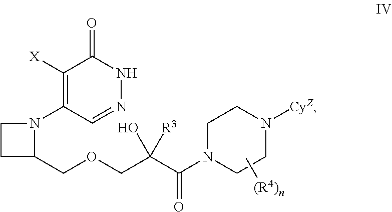

or a pharmaceutically acceptable salt thereof.

In some embodiments, the compound of Formula I has Formula V:

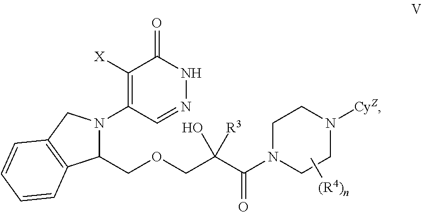

or a pharmaceutically acceptable salt thereof.

In some embodiments, the compound of Formula I has Formula VI:

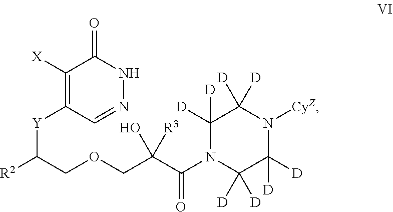

or a pharmaceutically acceptable salt thereof.

In some embodiments, provided herein is a compound of Formula I, or a pharmaceutically acceptable salt thereof, wherein:

X is halo or CF₃;

Y is NR¹ or O;

R¹ is H;

R² is H or C₁₋₆ alkyl, wherein said C₁₋₆ alkyl is optionally substituted with OR^{a};

or R¹ and R² together with the atoms to which they are attached form a 4-10 membered heterocycloalkyl group; wherein the 4-10 membered heterocycloalkyl group has at least one ring-forming carbon atom and 1, 2, or 3 ring-forming heteroatoms independently selected from N, O, and S;

R³ is H or C₁₋₆ alkyl;

each R⁴ is D;

Cy^{Z} is a 5-10 membered heteroaryl group substituted with R^{Z}.

R^{Z} is halo, CN, or C₁₋₃ haloalkyl;

R^{a} is selected from C₁₋₆ alkyl and C₁₋₆ haloalkyl; and

n is 0 or 8.

In some embodiments, provided herein is a compound selected from:

5-(((S)-1-((R)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one;

5-(((S)-1-((S)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one; 5-(((S)-1-((R)-2-hydroxy-3-oxo-3-(4-(5-(trifluoromethyl)pyrazin-2-yl)piperazin-1-yl)propoxy)propan-2-yl)amino)-4-(trifluoromethyl)pyridazin-3(2H)-one; 5-(((S)-1-((S)-2-hydroxy-3-oxo-3-(4-(5-(trifluoromethyl)pyrazin-2-yl)piperazin-1-yl)propoxy)propan-2-yl)amino)-4-(trifluoromethyl)pyridazin-3(2H)-one;

5-((S)-1-((R)-2-hydroxy-3-oxo-3-(4-(5-(trifluoromethyl)thiazol-2-yl)piperazin-1-yl)propoxy)propan-2-ylamino)-4-(trifluoromethyl)pyridazin-3(2H)-one; 5-((S)-1-((S)-2-hydroxy-3-oxo-3-(4-(5-(trifluoromethyl)thiazol-2-yl)piperazin-1-yl)propoxy)propan-2-ylamino)-4-(trifluoromethyl)pyridazin-3(2H)-one;

5-(((S)-1-((R)-3-(4-(5-(difluoromethyl)pyrimidin-2-yl)piperazin-1-yl)-2-hydroxy-3-oxopropoxy)propan-2-yl)amino)-4-(trifluoromethyl)pyridazin-3(2H)-one;

5-(((S)-1-((S)-3-(4-(5-(difluoromethyl)pyrimidin-2-yl)piperazin-1-yl)-2-hydroxy-3-oxopropoxy)propan-2-yl)amino)-4-(trifluoromethyl)pyridazin-3(2H)-one;

5-(((2S)-1-(2-hydroxy-2-methyl-3-oxo-3-(4-(5-(trifluoromethyl)pyrimidin-2-yl)piperazin-1-yl)propoxy)propan-2-yl)amino)-4-(trifluoromethyl)pyridazin-3(2H)-one;

5-(((S)-1-((S)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one; 5-(((S)-1-((R)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one;

5-(((S)-1-((S)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one;

5-(((S)-1-((R)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one;

5-(((S)-1-((R)-2-hydroxy-3-oxo-3-(4-(5-(trifluoromethyl)pyrazin-2-yl)piperazin-1-yl)propoxy)propan-2-yl)oxy)-4-(trifluoromethyl)pyridazin-3(2H)-one;

5-(((S)-1-((S)-2-hydroxy-3-oxo-3-(4-(5-(trifluoromethyl)pyrazin-2-yl)piperazin-1-yl)propoxy)propan-2-yl)oxy)-4-(trifluoromethyl)pyridazin-3(2H)-one;

5-(((S)-1-((R)-3-(4-(5-chloropyrimidin-2-yl)piperazin-1-yl)-2-hydroxy-3-oxopropoxy)propan-2-yl)oxy)-4-(trifluoromethyl)pyridazin-3(2H)-one;

6-(4-((R)-2-hydroxy-3-((S)-2-((6-oxo-5-(trifluoromethyl)-1,6-dihydropyridazin-4-yl)oxy)propoxy)propanoyl)piperazin-1-yl)nicotinonitrile; 5-(((S)-1-((R)-3-(4-(5-(difluoromethyl)pyrimidin-2-yl)piperazin-1-yl)-2-hydroxy-3-oxopropoxy)propan-2-yl)oxy)-4-(trifluoromethyl)pyridazin-3(2H)-one;

4-bromo-5-(((S)-1-((S)-2-hydroxy-3-oxo-3-(4-(5-(trifluoromethyl)pyrimidin-2-yl)piperazin-1-yl)propoxy)propan-2-yl)oxy)pyridazin-3(2H)-one;

15 4-bromo-5-(((S)-1-((R)-2-hydroxy-3-oxo-3-(4-(5-(trifluoromethyl)pyrimidin-2-yl)piperazin-1-yl)propoxy)propan-2-yl)oxy)pyridazin-3(2H)-one;

5-(((S)-1-((S)-2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-4-(trifluoromethyl)pyridazin-3(2H)-one;

5-(((S)-1-((R)-2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-4-(trifluoromethyl)pyridazin-3(2H)-one; 5-(((2S)-1-(2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one;

5-(((2S)-1-(2-hydroxy-3-oxo-3-(4-(5-(trifluoromethyl)pyrimidin-2-yl)piperazin-1-yl)propoxy)butan-2-yl)amino)-4-(trifluoromethyl)pyridazin-3(2H)-one;

25 5-(((R)-1-((R)-2-hydroxy-3-oxo-3-(4-(5-(trifluoromethyl)pyrimidin-2-yl)piperazin-1-yl)propoxy)-3-(trifluoromethoxy)propan-2-yl)amino)-4-(trifluoromethyl)pyridazin-3(2H)-one;

5-(((S)-1-((R)-2-hydroxy-3-oxo-3-(4-(5-(trifluoromethyl)pyrimidin-2-yl)piperazin-1-yl)propoxy)-3-(trifluoromethoxy)propan-2-yl)amino)-4-(trifluoromethyl)pyridazin-3(2H)-one;

5-((S)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;

5-((S)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;

5-((R)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;

5-((R)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one; 5-((2S)-2-((2-hydroxy-3-oxo-3-(4-(5-(trifluoromethyl)pyrimidin-2-yl)piperazin-1-yl)propoxy)methyl)azetidin-1-yl)-4-(trifluoromethyl)pyridazin-3(2H)-one; and 5-(((S)-1-((R)-2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one;

or a pharmaceutically acceptable salt of any of the aforementioned.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ.” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl.

As used herein, “halo” or “halogen”, employed alone or in combination with other terms, includes fluoro, chloro, bromo, and iodo. In some embodiments, halo is F or Cl.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the alkyl group has 1 to 6 or 1 to 4 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “dialkylamino,” employed alone or in combination with other terms, refers to a group of formula —N(alkyl)₂. Example dialkylamino groups include dimethylamino, diethylamino, dipropylamino (e.g., di(n-propyl)amino and di(isopropyl)amino), and the like. In some embodiments, each alkyl group independently has 1 to 6 or 1 to 4 carbon atoms.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahyropyridine, azetidine ring, isoindoline ring, or tetrahydrofuran ring.

As used herein, the term “aryl.” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridavinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has I to 4, 1 to 3, or 1 to 2 heteroatoms.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H- isoindole, and 1H- and 2H-pyrazole. Tautomeric forms can be in equilibrium or sterically locked into one form by appropriate substitution. An example of tautomeric forms, pyridazin-3(2H)-one and pyridazin-3-ol, is depicted below:

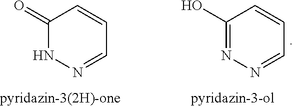 [SMILES: O=c1cccn[nH]1 ; Oc1cccnn1]

Compounds of the invention also include all isotopes of atoms occurring in the intermediates or final compounds. Isotopes include those atoms having the same atomic number but different mass numbers. For example, isotopes of hydrogen include tritium and deuterium. In some embodiments, the compounds of the invention include at least one deuterium atom.

The term, “compound,” as used herein is meant to include all stereoisomers, geometric iosomers, tautomers, and isotopes of the structures depicted, unless otherwise specified.

All compounds, and pharmaceutically acceptable salts thereof, can be found together with other substances such as water and solvents (e.g., in the form of hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

In some embodiments, the compounds of the invention, or their salts, can be prepared with substantial purity. For example, a preparation of a compound of the invention, or a pharmaceutically acceptable salt thereof, can have at least about 85%, at least about 90%, at least about 95%, at least about 98%, at least about 99%, or at least about 99.5% purity. Purity is generally expressed as the ratio (or percent) of mass of the compound (or pharmaceutically acceptable salt thereof) to total mass of the sample.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “RT”, as used herein, are understood in the art, and refer generally to a temperature, e.g. a reaction temperature, that is about the temperature of the room in which the reaction is carried out, for example, a temperature from about 20° C. to about 30° C.

Compounds of Formula I can be prepared according to numerous preparatory routes known in the literature. Example synthetic methods for preparing compounds of the invention are provided in the Schemes below. Unless noted otherwise, all substituents are as defined herein.

In the process depicted in Scheme 1, amine 1-1 can be coupled with compound 1-2 (wherein X^{a} is halogen) in the presence of a base (e.g., triethylamine) to provide compound 1-3. Compound 1-3 can be alkylated with compound 1-4 in the presence of a Lewis acid (e.g., BF3) to provide compound 1-5. Compound 1-5 can be saponified with a suitable base (e.g., LiOH, KOH, or NaOH) to provide compound 1-6. Compound 1-6 can be coupled with compound 1-7 under standard peptide coupling conditions (e.g., DIEA and HATU) to provide compound 1-8. Compound 1-8 can be deprotected with a suitable acid (e.g., triflic acid in TFA) to provide compound 1-9.

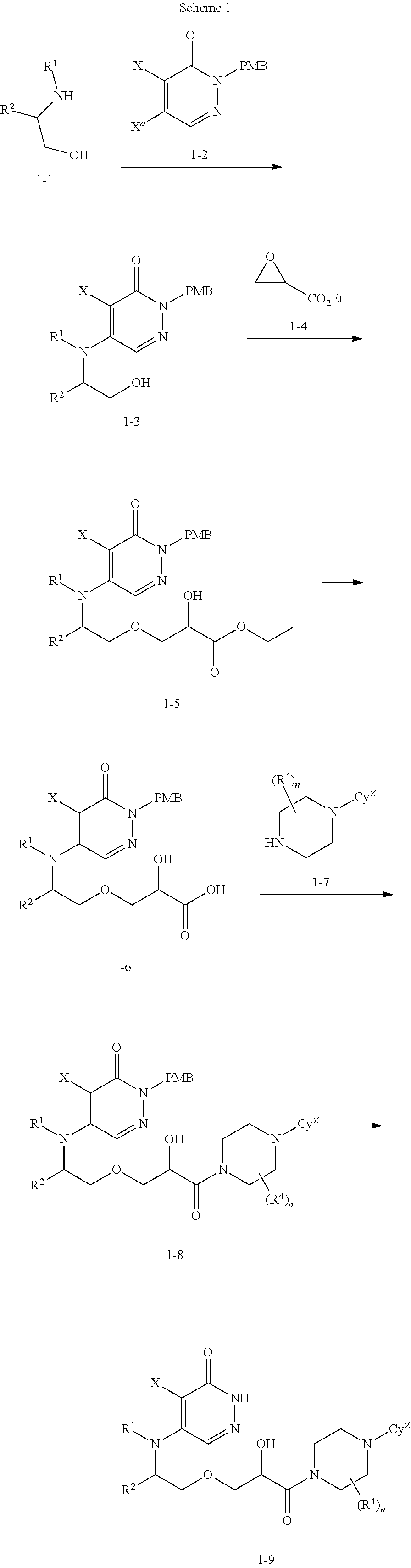

In the process depicted in Scheme 2, compound 2-1 can be coupled with compound 2-2 (wherein X^{a} is halogen) in the presence of a base (e.g., potassium tert-butoxide) to give compound 2-3. Compound 2-3 can be deprotected with an appropriate Lewis acid (e.g., BCl₃) to provide compound 2-4. Compound 2-4 can be alkylated with compound 2-5 in the presence of a Lewis acid (e.g., Mg(ClO₄)₂) to provide compound 2-6. Compound 2-6 can be saponified with a suitable base (e.g., LiOH, KOH, or NaOH) to provide compound 2-7. Compound 2-7 can be coupled with compound 2-8 under standard peptide coupling conditions (e.g., DIEA and HATU) to provide compound 2-9. Compound 2-9 can be deprotected with a suitable acid (e.g., triflic acid in TFA) to provide compound 2-10.

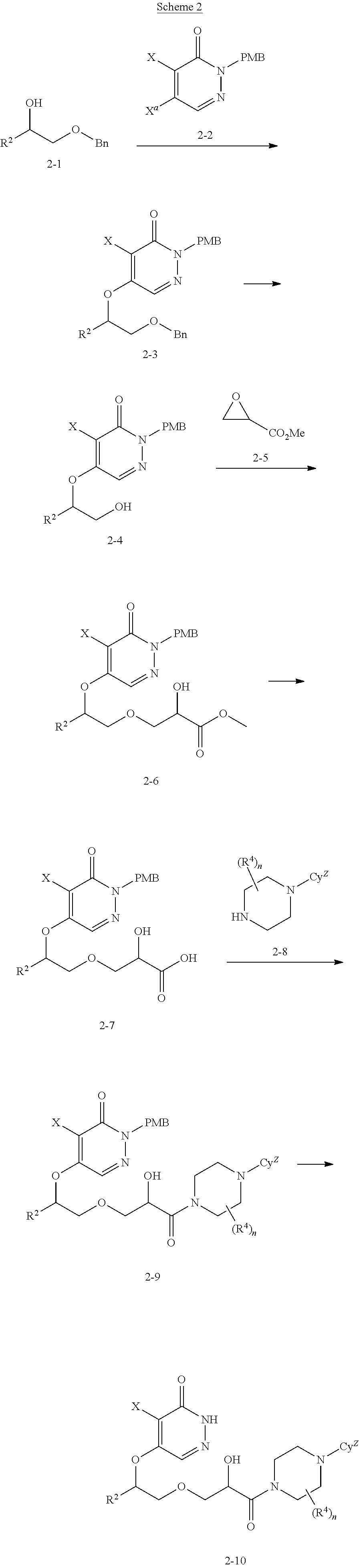

In the process depicted in Scheme 3. compound 3-1 can be oxidized to compound 3-2 with a suitable oxidant (e.g., Dess-Martin periodinane). Compound 3-2 can then be treated with an appropriately substituted nucleophilic reagent (e.g., R³MgBr) to provide compound 3-3.

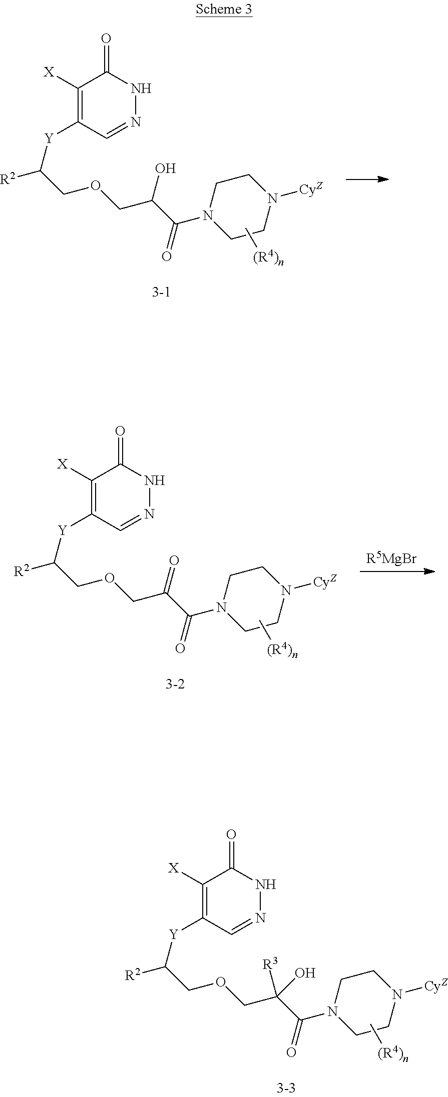

Scheme 4 illustrates a process for obtaining compounds of Formula 1-2 (see Scheme 1) or 2-2 (see Scheme 2). An appropriately substituted, halogen containing compound (i.e., X^{a}=Cl or Br) of Formula A is protected as the p-methoxy benzyl ether (“PMB”) compound of Formula 1-2 (or 2-2) by treatment with p-methoxy benzyl chloride (“PMB-CI”) in the presence of sodium hydride (NaH).

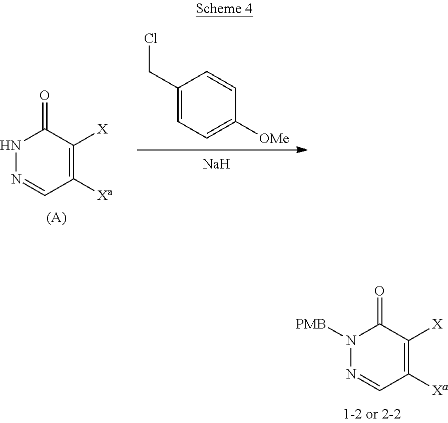

### Methods of Use

Compounds of the invention can inhibit the activity of PARP7. For example, the compounds of the invention can be used to inhibit activity of PARP7 in a cell or in an individual or patient in need of inhibition of the enzyme by administering an inhibiting amount of a compound of the invention to the cell, individual, or patient.

As PARP7 inhibitors, the compounds of the invention are useful in the treatment of various diseases associated with abnormal expression or activity of PARP7. For example, the compounds of the invention are useful in the treatment of cancer. In some embodiments, the cancers treatable according to the present invention include breast, central nervous system, endometrium, kidney, large intestine, lung, oesophagus, ovary, pancreas, prostate, stomach, head and neck (upper aerodigestive), urinary tract, colon, and others.

In some embodiments, the cancers treatable according to the present invention include hematopoietic malignancies such as leukemia and lymphoma. Example lymphomas include Hodgkin's or non-Hodgkin's lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett's lymphoma. Example leukemias include acute lymphocytic leukemia (ALL), acute myelogenous leukemia (AML), chronic lymphocytic leukemia (CLL), and chronic myelogenous leukemia (CML).

Other cancers treatable by the administration of the compounds of the invention include liver cancer (e.g., hepatocellular carcinoma), bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, head and neck cancer (upper aerodigestive cancer), intestinal cancers, Kaposi's sarcoma, kidney cancer, laryngeal cancer, liver cancer (e.g., hepatocellular carcinoma), lung cancer, prostate cancer, rectal cancer, skin cancer, stomach cancer, testicular cancer, thyroid cancer, and uterine cancer.

In some embodiments, the cancer treatable by administration of the compounds of the invention is multiple myeloma, DLBCL, hepatocellular carcinoma, bladder cancer, esophageal cancer, head and neck cancer (upper aerodigestive cancer), kidney cancer, prostate cancer, rectal cancer, stomach cancer, thyroid cancer, uterine cancer, and breast cancer.

The PARP7 inhibitors of the invention may also have therapeutic utility in PARP7-related disorders in disease areas such as cardiology, virology, neurodegeneration, inflammation, and pain, particularly where the diseases are characterized by overexpression or increased activity of PARP7.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PARP7 or “contacting” a cell with a compound of the invention includes the administration of a compound of the present invention to an individual or patient, such as a human, having PARP7, as well as, for example, introducing a compound of the invention into a sample containing a cellular or purified preparation containing PARP7.

As used herein, the term “individual” or “patient,” used interchangeably, refers to mammals, and particularly humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease.

### Combination Therapy

One or more additional pharmaceutical agents or treatment methods such as, for example, chemotherapeutics or other anti-cancer agents, immune enhancers, immunosuppressants, immunotherapies, radiation, anti-tumor and anti-viral vaccines, cytokine therapy (e.g., IL2, GM-CSF, etc.), and/or kinase (tyrosine or serine/threonine), epigenetic or signal transduction inhibitors can be used in combination with the compounds of the present invention. The agents can be combined with the present compounds in a single dosage form, or the agents can be administered simultaneously or sequentially as separate dosage forms.

Suitable agents for use in combination with the compounds of the present invention for the treatment of cancer include chemotherapeutic agents, targeted cancer therapies, immunotherapies or radiation therapy. Compounds of this invention may be effective in combination with anti-hormonal agents for treatment of breast cancer and other tumors.

Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™M), ifosfamide, melphalan, chlorambucil, pipobroman, triethylene-melamine, triethylenethiophosphoramine, busulfan, carmustine, lomustine, streptozocin, dacarbazine, and temozolomide.

Other anti-cancer agent(s) include antibody therapeutics to checkpoint or costimulatory molecules such as CTLA-4, PD-1, PD-L1 or 4-1BB, respectively, or antibodies to cytokines (IL-10, TGF-β, etc.). Exemplary cancer immunotherapy antibodies include pembrolizumab, ipilimumab, nivolumab, atezolizumab and durvalumab. Additional anti-cancer agent(s) include antibody therapeutics directed to surface molecules of hematological cancers such as ofatumumab, rituximab and alemtuzumab.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

Pharmaceutical Formulations and Dosage Forms When employed as pharmaceuticals, the compounds of the invention can be administered in the form of pharmaceutical compositions. A pharmaceutical composition refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, anti-cancer agents, vaccines, antibodies, immune enhancers, immune suppressants, anti-inflammatory agents and the like.

### EXAMPLES

### Equipment

¹H NMR Spectra were recorded at 300 or 400 MHz using a Bruker AVANCE 300 MHz/400 MHz spectrometer. NMR interpretation was performed using Bruker Topspin software to assign chemical shift and multiplicity. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks. LCMS equipment and conditions are as follows:
   1. LC (basic condition): Shimadzu LC-20AD, Binary Pump, Diode Array Detector. Column: Kinetex 2.6 μm EVO C18 100A, 50*3.0 mm, 2.6 μm. Mobile phase: A: Water/5 mM NH₄HCO₃, B: Acetonitrile. Flow Rate: 1.2 mL/min at 40° C. Detector: 254 nm, 220 nm. Gradient stop time, 2.9 min. Timetable:

| T (min) | A(%) | B(%) |
|---|---|---|
| 0.01 | 90 | 10 |
| 2.10 | 5 | 95 |
| 2.70 | 5 | 95 |
| 2.90 | 90 | 10 |

2. LC (basic condition): Shimadzu LC-20ADXR, Binary Pump, Diode Array Detector, Column: Poroshell HPH-C18 50*3.0 mm, 2.7 μm. Mobile Phase A: 0.04% Ammonium hydroxide, Mobile Phase B: Acetonitrile. Flow Rate: 1.2 ml/min at 40° C. Detector: 254 nm, 220 nm. Gradient stop time 3.0 min. Timetable:

| T(min) | A(%) | B(%) |
|---|---|---|
| 0.01 | 90 | 10 |
| 2.0 | 5 | 95 |
| 2.7 | 5 | 95 |
| 2.8 | 90 | 10 |

3. LC (acidic condition): Shimadzu LC-20AD, Binary Pump, Diode Array Detector. Column: Ascentis Express C18, 50*3.0 mm, 2.7 μm. Mobile phase: A: Water/0.05% TFA, B: Acetonitrile/0.05% TFA. Flow Rate: 1.5 mL/min at 40° C. Detector: 254 nm, 220 nm. Gradient stop time, 2.9 min. Timetable:

| T (min) | A(%) | B(%) |
|---|---|---|
| 0.01 | 90 | 5 |
| 2.10 | 5 | 95 |
| 2.70 | 5 | 95 |
| 2.90 | 90 | 5 |

4. LC (acidic condition): Shimadzu LC-30AD, Binary Pump, Diode Array Detector. Column: Accucore C18 50*2.1 mm, 2.6 μm. Mobile Phase A: Water/0.1% FA Mobile Phase B: Acetonitrile/0.1% FA. Flow Rate: 1.0 ml/min at 40° C. Detector: 254 nm, 220 nm. Gradient stop time 3.0 min. Timetable:

| T(min) | A(%) | B(%) |
|---|---|---|
| 0.01 | 95 | 5 |
| 2.0 | 5 | 95 |
| 2.7 | 5 | 95 |
| 2.8 | 95 | 5 |

1. S:LCMS-2020, Quadrupole LC/MS, Ion Source: ES-API, TIC: 90˜900 m/z, Fragmentor: 60, Drying gas flow: 15 L/min, Nebulizing Gas Flow: 1.5 L/min, Drying gas temperature: 250° C., Vcap: 1100V.
   2. Sample preparation: samples were dissolved in ACN or methanol at 1˜10 mg/mL, then filtered through a 0.22 μm filter membrane. Injection volume: 1˜10 μL.
Definitions: ACN (acetonitrile); AgOTf (silver triflate); BCl₃ (boron trichloride): BF_{3:}Et₂O (boron trifluoride etherate); CF₃CN (acetonitrile); CDCl₃ (deuterated chloroform); CD₃OD (deuterated methanol); Cs₂CO₃ (cesium carbonate); DCE (1,2-dichloroethane), DCM (dichloromethane); DEA (diethylamine); DIEA (diisopropylethyl amine); DMF (N,N-dimethylformamide); DMAP (4-dimethyl aminopyridine); DMSO (dimethylsulfoxide); DMSO-d₆ (deuterated dimethylsulfoxide); equiv (equivalent); ESI (electrospray ionization); EtOAc (ethyl acetate); EtOH (ethanol); FA (formic acid); g (gram); h (hour); HATU (1-[bis(dimethylamino)methylene]-1H-1,2,3-triazolo[4,5-b]pyridinium 3-oxide hexafluorophosphate); ¹H NMR (proton nuclear magnetic resonance); H₂O (water); HCl (hydrochloric acid); Hz (hertz); Hex (hexanes); i-prOH (isopropyl alcohol); K₂CO₃ (potassium carbonate); KF (potassium fluoride); L (litre); LAH (lithium aluminum hydride); LCMS (liquid chromatography-mass spectrometry); LiOH H₂O (lithium hydroxide hydrate); M (molar); MeCN (acetonitrile); MeOH (methanol); MesSn(OH) (trimethyl tin hydroxide); mg (milligrams); MHz (megahertz); min (minutes); Mg(ClO₄)₂ (magnesium perchlorate); MtBE (methyl tert-butyl ether); mL (millilitres), mmol (millimoles); N (normal); NaCl (sodium chloride); NaH (sodium hydride); NaHCO₃ (sodium bicarbonate); Na₂CO; (sodium carbonate); Na₂SO₄ (sodium sulfate); n-BuOH (n-butyl alcohol); NH₃ (ammonia); NH₄Cl (ammonium chloride); NMP (N-methyl-2-pyrrolidone); PMB (para-methoxy benzyl) prep-HPLC (preparative high-performance liquid chromatography); RT (room temperature); t-BuONa (sodium tert-butoxide); TEA (triethylamine); TFA (trifluoroacetic acid); TMSCF₃ (trifluoromethyl trimethylsilane); tR (retention time); triflic (trifluoromethane sulfonic).
   Intermediate I-1: Synthesis of 5-Chloro-2-(4-methoxybenzyl)-4-(trifluoromethyl)pyridazin-3(2H)-one

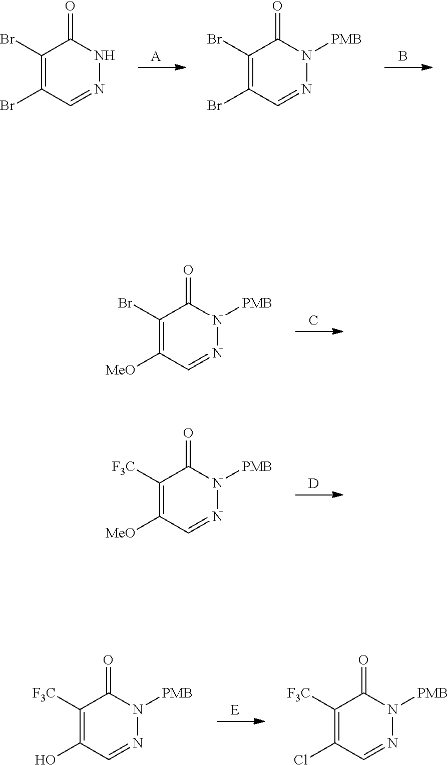

### Step A

To a solution of 4,5-dibromo-2,3-dihydropyridazin-3-one (250 g, 984.71 mmol, 1 equiv) in DMF (2.5 L) was added NaH (59.1 g, 1477.07 mmol, 1.50 equiv, 60%) in several batches at 0-10° C. followed by the addition of 1-(chloromethyl)-4-methoxy benzene (230.3 g, 1470.53 mmol, 1.49 equiv) at 0° C. The resulting solution was stirred for 3 h at RT. The reaction was then quenched by the addition of 5 L of water/ice and extracted with 2×2.5 L of DCM. The organic layers were combined and concentrated. The solids were washed by MeOH (500 mL×2) to afford 290 g (79% yield) of 4,5-dibromo-2-[(4-methoxyphenyl)methyl]-2,3-dihydropyridazin-3-one as a solid. LCMS [M+H]⁺378.00.

### Step B

A solution of 4,5-dibromo-2-[(4-methoxyphenyl)methyl]-2,3-dihydropyridazin-3-one (290 g, 775.33 mmol, 1 equiv), potassium hydroxide (130.5 g, 2326.00 mmol, 3.00 equiv) in MeOH (2.5 L) was stirred for 2 h at RT. The resulting mixture was concentrated to 500 mL and the solids were collected by filtration. The resulting cake was slurried for 1 h in water (IL) to afford 232 g (92% yield) of 4-bromo-5-methoxy-2-[(4-methoxyphenyl)methyl]-2,3-dihydropyridazin-3-one as a solid. LCMS [M+H]⁺326.90.

### Step C

A solution of 4-bromo-5-methoxy-2-[(4-methoxyphenyl)methyl]-2,3-dihydropyridazin-3-one (232 g, 713.49 mmol, 1 equiv), methyl 2,2-difluoro-2-sulfoacetate (411.2 g, 2140.44 mmol, 3.00 equiv), and Cul (67.9 g, 356.52 mmol, 0.50 equiv) in NMP (1.2L) was stirred for 3 h at 100° C. The reaction was then quenched by the addition of 1.5L of water. The resulting solution was extracted with 3×1 L of DCM. The organic layers were combined and concentrated. The residue was applied onto a silica gel column with EtOAc/petroleum ether (1/1). The collected fractions were combined and concentrated to afford the crude oil to which was added IL of water. The solids were collected by filtration and washed with 100 mL of MeOH to afford 170 g (76% yield) of 5-methoxy-2-[(4-methoxy phenyl)methyl]-4-(trifluoromethyl)-2,3-dihydropyridazin-3-one as a solid. LCMS [M+H]⁺315.10.

### Step D

To a solution of 5-methoxy-2-[(4-methoxyphenyl)methyl]-4-(trifluoromethyl)-2,3-dihydropyridazin-3-one (170 g, 540.95 mmol, 1 equiv) in DMF (850 mL) was added TMSI (140 g, 699.67 mmol, 1.29 equiv) dropwise at 20° C. The resulting solution was stirred for 20 h at 85° C. The reaction mixture was then quenched by the addition of 850 ml of water and the resulting solution was extracted with 3×850 mL of DCM and the organic layers combined and dried over anhydrous sodium sulfate. The organic layers were concentrated under vacuum and the crude product was purified by silica gel column chromatography and then recrystallized with MtBE to afford 120 g (74% yield) of 5-hydroxy-2-[(4-methoxy phenyl)methyl]-4-(trifluoromethyl)-2,3-dihydropyridazin-3-one as a white solid. LCMS [M+H]⁺301.07.

### Step E

To a solution of 5-hydroxy-2-[(4-methoxyphenyl)methyl]-4-(trifluoromethyl)-2,3-dihydropyridazin-3-one (110 g, 366.38 mmol, 1 equiv) in DMF (550 mL) was added oxalic dichloride (93 g, 732.75 mmol, 2.00 equiv) dropwise at 0-5° C. The resulting solution was stirred for 8 h at RT. The reaction was then quenched by the addition of 550 ml of water. The solids were collected by filtration to afford 108 g (93%) of 5-chloro-2-[(4-methoxyphenyl)methyl]-4-(trifluoromethyl)-2,3-dihydropyridazin-3-one as a white solid. LCMS [M+H]⁺319.04 [M+H]⁺, ¹H NMR (30 MHz, DMSO-d₆) δ8.22 (d, J=0.8 Hz, 1H), 7.33-7.22 (m, 2H), 6.94-6.84 (m, 2H), 5.18 (s, 2H), 3.71 (s, 3H).

Intermediate I-2: Synthesis of 2-hydroxy-3-((S)-2-((1-(4-methoxybenzyl)-6-oxo-5-(trifluoromethyl)-1,6-dihydropyridazin-4-yl)oxy)propoxy)propanoic Acid

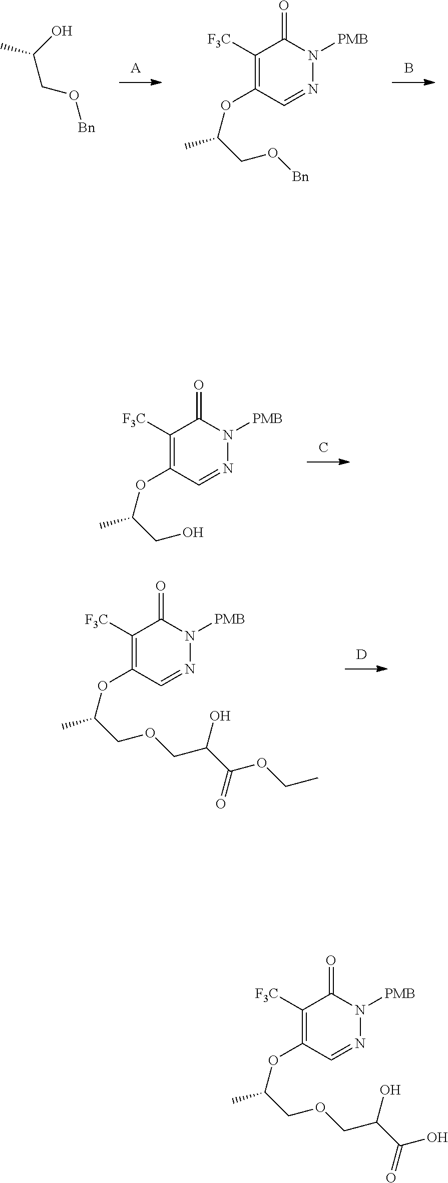

### Step A

A solution of 5-chloro-2-(4-methoxy benzyl)-4-(trifluoromethyl)pyridazin-3(2H)-one (10.0 g, 31.4 mmol, 1.0 equiv), (S)-1-(benzyloxy)propan-2-ol (5.22 g, 31.4 mmol, 1.0 equiv) and sodium tert-butoxide (3.00 g, 31.2 mmol, 0.99 equiv) in DCM (150 mL) was stirred for 1 h at 0° C. in an ice bath. After filtration, the filtrate was concentrated under vacuum and the crude product was applied onto a silica gel column eluting with ethyl acetate/petroleum ether (30:70) to afford the (S)-5-((1-(benzyloxy)propan-2-yl)oxy)-2-(4-methoxy benzyl)-4-(trifluoromethyl)pyridazin-3(2H)-one (8.0 g, 57% yield) as a yellow oil. LCMS (ESI, m/z): 449.15 [M+H]⁺.

### Step B

To a solution of (S)-5-((1-(benzyloxy)propan-2-yl)oxy)-2-(4-methoxy benzyl)-4-(trifluoromethyl)pyridazin-3(2H)-one (4.80 g, 10.7 mmol, 1.0 equiv) in DCM (100 mL) at 0° C. was added BCl3 (1 M in DCM; 32 mL, 3.0 equiv) dropwise. The resulting solution was stirred for 1 h at 0° C. at which point ice water (100 mL) was added to the reaction mixture. The resulting solution was extracted with 2×300 mL of dichloromethane. The organic layers were combined, dried over anhydrous sodium sulfate and concentrated under vacuum. The crude product was applied onto a silica gel column eluting with ethyl acetate/petroleum ether (20:80) to afford (S)-5-((1-hydroxypropan-2-yl)oxy)-2-(4-methoxybenzyl)-4-(trifluoromethyl)pyridazin-3(2H)-one (2.1 g, 55% yield) as a yellow solid. LCMS (ESI, m/z): 359.10 [M+H]⁺.

### Step C

A solution of (S)-5-((1-hydroxypropan-2-yl)oxy)-2-(4-methoxy benzyl)-4-(trifluoromethyl)pyridazin-3(2H)-one (2.70 g, 7.535 mmol, 1.0 equiv), Mg(CIO4)2 (2.52 g, 11.3 mmol, 1.5 equiv) and ethyl oxirane-2-carboxylate (1.14 g, 9.8 mmol, 1.3 equiv) in EtOAc (25 mL) was stirred for 2 days at 80° C. After filtration, the filtrate was concentrated. The crude product was applied onto a reverse phase column eluting with H₂O/CH₃CN (43/57) to afford ethyl 2-hydroxy-3-((S)-2-((1-(4-methoxy benzyl)-6-oxo-5-(trifluoromethyl)-1,6-dihydropyridazin-4-yl)oxy)propoxy)propanoate (866 mg, 24% yield) as a yellow oil. LCMS (ESI, m/z): 475.25 [M+H]⁺.

### Step D

A solution of ethyl 2-hydroxy-3-((S)-2-((1-(4-methoxybenzyl)-6-oxo-5-(trifluoromethyl)-1,6-dihydropyridazin-4-yl)oxy)propoxy)propanoate (1.50 g, 3.3 mmol, 1.0 equiv) and MesSn(OH) (2.36 g, 13.0 mmol, 4.0 equiv) in DCE (15 mL) was stirred for 2 h at 100° C. Water (50 mL) was added and the resulting solution was extracted with 2×150 ml of dichloromethane. The organic layers were combined, dried over anhydrous sodium sulfate and concentrated under vacuum. The crude product was applied onto a reverse phase column eluting with H₂O/CH₃CN (69/31) to afford 2-hydroxy-3-((S)-2-((1-(4-methoxybenzyl)-6-oxo-5-(trifluoromethyl)-1,6-dihydropyridazin-4-yl)oxy)propoxy)propanoic acid (573 mg, 39% yield) as a yellow oil. LCMS (ESI, m/z): 447.05 [M+H]⁺.

Intermediate I-3: Synthesis of 2-hydroxy-3-((S)-2-((1-(4-methoxybenzyl)-6-oxo-5-(trifluoromethyl)-1,6-dihydropyridazin-4-yl)amino)propoxy)propanoic Acid

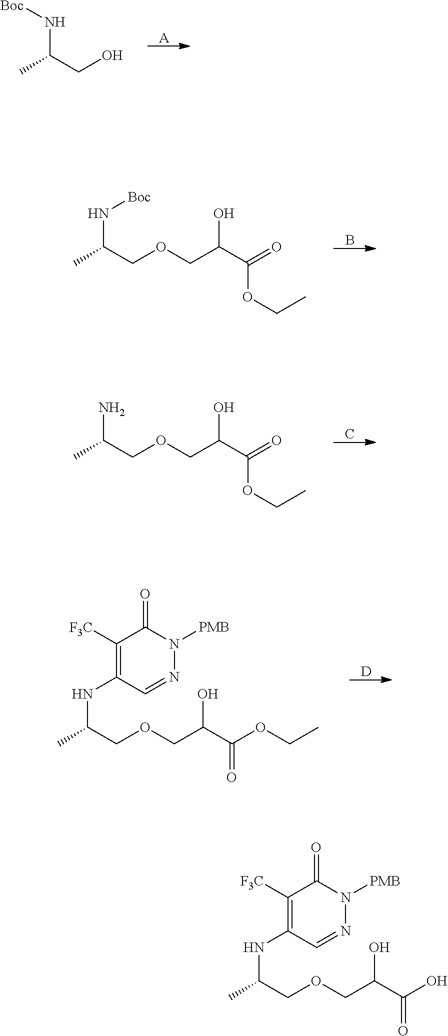

### Step A

A solution of tert-butyl (S)-(1-hydroxypropan-2-yl)carbamate (10.0 g, 57.1 mmol, 1.0 equiv), Mg(CIO4)2 (25.4 g. 114.1 mmol, 2.0 equiv) and ethyl oxirane-2-carboxylate (9.9 g, 85.6 mmol, 1.5 equiv) in EtOAc (200 mL) was stirred for 3 days at 60° C. 100 ml of water was added, the layers were separated, and the resulting aqueous solution was extracted with 3×100 mL of ethyl acetate. The organic layers were combined, dried over anhydrous sodium sulfate and concentrated under vacuum. The crude product was applied onto a silica gel column eluting with ethyl acetate/petroleum ether (2:8) to afford 11 g of impure ethyl 3-((S)-2-((tert-butoxy carbonyl)amino)propoxy)-2-hydroxypropanoate as a yellow oil contaminated with unreacted epoxide starting material. Carried on without further purification. LCMS (ESI, m/z): 292.2 [M+H]⁺.

### Step B

A solution of impure ethyl 3-((S)-2-((tert-butoxycarbonyl)amino)propoxy)-2-hydroxypropanoate (11.0 g, 37.8 mmol, 1.0 equiv) in DCM (20 mL) and trifluoroacetic acid (15 mL) was stirred for 2 hours at room temperature. The resulting mixture was concentrated to afford 12 g of ethyl 3-((S)-2-aminopropoxy)-2-hydroxypropanoate as a red oil that was carried on without further purification. LCMS (ESI, m/z): 192.1 [M+H]⁺.

### Step C

A solution of ethyl 3-((S)-2-aminopropoxy)-2-hydroxypropanoate (12.0 g, 39.3 mmol, 1.0 equiv), TEA (11.9 g, 117.9 mmol, 3.0 equiv) and 5-chloro-2-(4-methoxy benzyl)-4-(trifluoromethyl)pyridazin-3(2H)-one (6.26 g, 19.7 mmol, 0.5 equiv) in EtOH (200 mL) was stirred for 2 hours at 60° C. The mixture was concentrated under vacuum and the crude product was applied onto a silica gel column eluting with ethyl acetate/petroleum ether (7:3) to afford 6 g (crude) of ethyl 2-hydroxy-3-((S)-2-((1-(4-methoxy benzyl)-6-oxo-5-(trifluoromethyl)-1,6-dihydropyridazin-4-yl)amino)propoxy)propanoate as a yellow oil. LCMS (ESI, m/z): 474.2 [M+H]⁺.

### Step D

To a solution of ethyl 2-hydroxy-3-((S)-2-((1-(4-methoxy benzyl)-6-oxo-5-(trifluoromethyl)-1,6-dihydropyridazin-4-yl)amino)propoxy)propanoate (6.0 g, 12.7 mmol, 1.0 equiv) in MeOH (50 mL) and H₂O (15 mL) was added LiOH H₂O (1.6 g, 38.0 mmol, 3.0 equiv) portionwise at 0° C. The resulting solution was stirred for 1 hour at 0° C.and then the pH value of the solution was adjusted to pH 6 with 6N HCl. The mixture was concentrated and applied onto a silica gel column eluting with dichloromethane/methanol (95:5) to afford 1.6 g (6% yield, 4 steps) of 2-hydroxy-3-((S)-2-((1-(4-methoxybenzyl)-6-oxo-5-(trifluoromethyl)-1,6-dihydropyridazin-4-yl)amino)propoxy)propanoic acid as a yellow oil. LCMS (ESI, m/z): 446.1 [M+H]⁺.

Intermediate I-4: Synthesis of tert-butyl (R)-(1-hydroxy-3-(trifluoromethoxy)propan-2-yl)carbamate

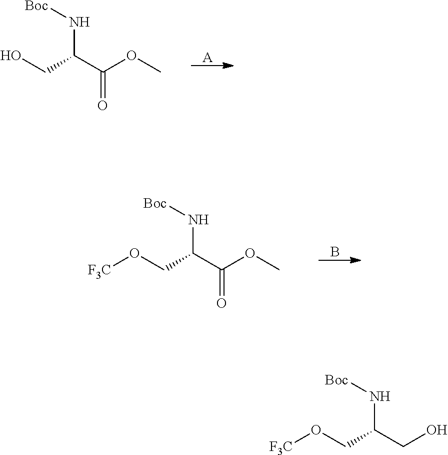

### Step A

A solution of methyl (tert-butoxy carbonyl)-L-serinate (24.0 g, 109 mmol, 1.0 equiv), 2-fluoropyridine (31.89 g, 328 mmol, 3.0 equiv), KF (25.44 g, 438 mmol, 4.0 equiv), TMSCF₃ (23.35 g, 164 mmol, 1.5 equiv), Selectfluor (116.34 g, 328 mmol, 3.0 equiv), and AgOTf (28.13 g, 109 mmol, 1.0 equiv) in EtOAc (400 mL) was stirred for 2.5 days at room temperature. The solids were filtered off and the filtrate was concentrated. The crude product was purified by silica gel chromatography eluting with ethyl acetate/petroleum ether (4/96) to afford 5.55 g (18%) of methyl N-(tert-butoxycarbonyl)-O-(trifluoromethyl)-L-serinate as a yellow oil. LCMS (ESI, m/z): 288.1 [M+H]⁺.

### Step B

To solution of methyl N-(tert-butoxycarbonyl)-O-(trifluoromethyl)-L-serinate (5.55 g, 19.3 mmol, 1.0 equiv) in THF (60 mL) at 0° C. was added LAH (2.20 g, 57.966 mmol, 3.00 equiv). After stirring for 1 hour at 0° C. the solution was treated carefully with water (2 mL), NaOH (2 mL, 15%), and then additional water (6mL). The resulting mixture was diluted with EtOAc (300 mL) and dried over anhydrous sodium sulfate. The solids were filtered off and the filtrate was concentrated. The crude product was applied onto a silica gel column with ethyl acetate/petroleum ether (1/4) to afford 4.0 grams (78% yield) of tert-butyl (R)-(1-hydroxy-3-(trifluoromethoxy)propan-2-yl)carbamate as a yellow oil. LCMS (ESI, m/z): 260.1 [M+H]⁺.

Intermediate I-5: Synthesis of (R)-2-hydroxy-3-((S)-2-((1-(4-methoxybenzyl)-6-oxo-5-(trifluoromethyl)-1,6-dihydropyridazin-4-yl)oxy)propoxy)propanoic Acid

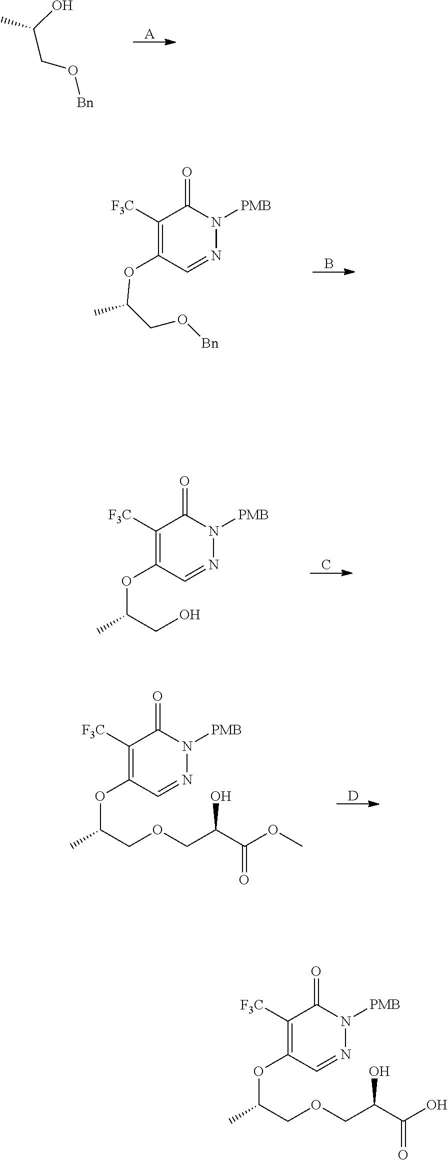

### Step A

A solution of 5-chloro-2-(4-methoxybenzyl)-4-(trifluoromethyl)pyridazin-3(2H)-one (3.83 g, 12.032 mmol, 1.00 equiv), (S)-1-(benzyloxy)propan-2-ol (2.00 g, 12.032 mmol, 1.00 equiv) and t-BuONa (1.16 g, 12.032 mmol, 0.99 equiv) in DCM (40 mL) was stirred for 1 h at 0° C. After filtration, the filtrate was concentrated under vacuum and the crude product was applied onto a silica gel column eluting with ethyl acetate/petroleum ether (30:70) to afford (S)-5-((1-(benzyloxy)propan-2-yl)oxy)-2-(4-methoxybenzyl)-4-(trifluoromethyl)pyridazin-3(2H)-one (4.28 g, 71% yield) as a yellow oil. LCMS: 449.15 [M+H]⁺.

### Step B

To a solution of (S)-5-((1-(benzyloxy)propan-2-yl)oxy)-2-(4-methoxybenzyl)-4-(trifluoromethyl)pyridazin-3(2H)-one (4.39 g, 9.789 mmol, 1.00 equiv) in DCM (40 mL) at 0° C. was added BCl₃ (1 M in DCM, 15 mL, 1.5 equiv) dropwise. The solution was maintained at that temperature for 1 hour and then 100 mL of ice water was added. The resulting solution was extracted with 2×300 mL of dichloromethane. The organic layers were combined, dried over anhydrous sodium sulfate, filtered and concentrated under vacuum. The crude product was applied onto a silica gel column eluting with ethyl acetate/petroleum ether (20:80) to afford (S)-5-((1-hydroxypropan-2-yl)oxy)-2-(4-methoxybenzyl)-4-(trifluoromethyl)pyridazin-3(2H)-one (1.57 g, 44% yield) as a yellow solid. LCMS (ESI, m/z): 359.10 [M+H]⁺.

### Step C

A solution of (S)-5-((1-hydroxypropan-2-yl)oxy)-2-(4-methoxy benzyl)-4-(trifluoromethyl)pyridazin-3(2H)-one (5.00 g, 13.954 mmol, 1.00 equiv), Mg(ClO₄)₂ (4.67 g, 20.931 mmol, 1.5 equiv) and methyl (R)-oxirane-2-carboxylate (1.85 g, 18.140 mmol, 1.3 equiv) in EtOAc (100 mL) was stirred for 2 days at 80° C. The resulting solution was concentrated. The crude product was applied onto a reverse phase column eluting with H₂O/CH₃CN (40/60) to afford ethyl (R)-2-hydroxy-3-((S)-2-((1-(4-methoxybenzyl)-6-oxo-5-(trifluoromethyl)-1,6-dihydropyridazin-4-yl)oxy)propoxy)propanoate (2.69 g, 22% yield) as a yellow oil. LCMS (ESI, m/z): 461.10 [M+H]⁺.

### Step D

A solution of ethyl (R)-2-hydroxy-3-((S)-2-((1-(4-methoxy benzyl)-6-oxo-5-(trifluoromethyl)-1,6-dihydropyridazin-4-yl)oxy)propoxy)propanoate (1.50 g, 3.258 mmol, 1.00 equiv), and MesSn(OH) (2.36 g, 13.032 mmol, 4.00 equiv) in DCE (15 mL) was stirred for 2 hours at 100° C. Water (50 mL) was added and the resulting solution was extracted with 2×150 mL of dichloromethane. The organic layers were combined, dried over anhydrous sodium sulfate and concentrated under vacuum. The crude product was purified by reverse phase chromatography eluting with H₂O/CH₃CN (69/31) to afford (R)-2-hydroxy-3-((S)-2-((1-(4-methoxybenzyl)-6-oxo-5-(trifluoromethyl)-1,6-dihydropyridazin-4-yl)oxy)propoxy)propanoic acid (573 mg, 39% yield) as a yellow oil. LCMS (ESI, m/z): 447.05 [M+H]⁺.

### Example 1: Synthesis of 5-(((S)-1-((R)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (1A) and 5-(((S)-1-((S)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (1B)

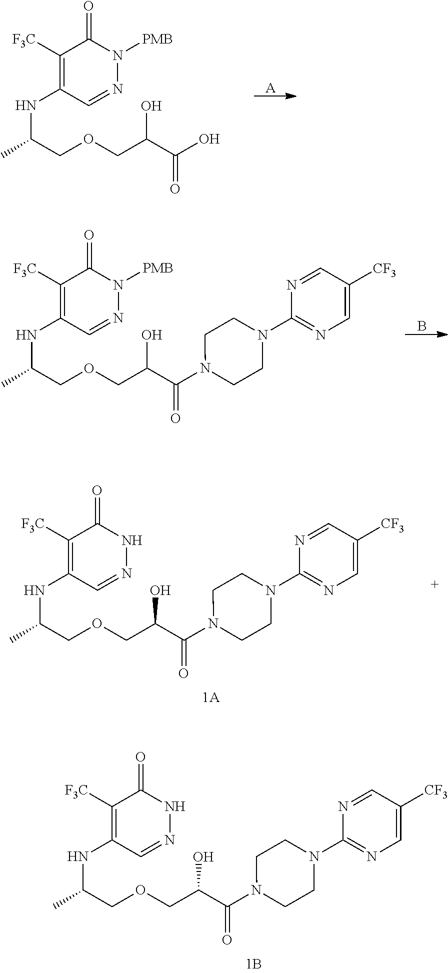

### Step A

A solution of 2-hydroxy-3-[(2S)-2-([1-[(4-methoxyphenyl)methyl]-6-oxo-5-(trifluoromethyl)pyridazin-4-yl]amino)propoxy ]propanoic acid (1.60 g, 3.6 mmol, 1.0 equiv), 2-(piperazin-1-yl)-5-(trifluoromethyl)pyrimidine (917 mg, 3.95 mmol, 1.10 equiv), DIEA (1.39 g, 10.8 mmol, 3.0 equiv) and HATU (2.05 g, 5.4 mmol, 1.5 equiv) in DMF (20 mL) was stirred for 1 h at room temperature. After completion, the reaction was quenched by the addition of 50 ml of water. The resulting solution was extracted with 3×100 ml of ethyl acetate. The organic layers were combined, washed with 3×50 ml of saturated sodium chloride aqueous solution, dried over Na₂SO₄, and then concentrated under vacuum. The residue was applied onto a silica gel column eluting with ethyl acetate/petroleum ether (4:1) to afford 2.1 g (89%) of 5-[[(2S)-1-(2-hydroxy-3-oxo-3-[4-[5-(trifluoromethyl)pyrimidin-2-yl]piperazin-1-yl]propoxy)propan-2-yl]amino]-2-[(4-methoxyphenyl)methyl]-4-(trifluoromethyl)pyridazin-3-one as a yellow solid. LC-MS: (ES, m z): 660.2 [M+H]⁺.

### Step B

To a solution of 5-[[(2S)-1-(2-hydroxy-3-oxo-3-[4-[5-(trifluoromethyl)pyrimidin-2-yl]piperazin-1-yl]propoxy)propan-2-yl]amino]-2-[(4-methoxyphenyl)methyl]-4-(trifluoromethyl)pyridazin-3-one (3.2 g, 4.9 mmol, 1.0 equiv) in TFA (30 mL) at 0° C. was added triflic acid (3 mL). The resulting solution was stirred for 1 h at 0° C. and then the pH value of the solution was adjusted to 8 with aqueous saturated sodium carbonate solution. The solution was extracted with 3×50 mL of dichloromethane. The organic layers were concentrated under vacuum and the crude product was purified by silica gel chromatography eluting with dichloromethane/methanol (98:2) to give a mixture of diastereomers. The mixture was separated by chiral prep-HPLC with following conditions: CHIRALPAK IG, 3*25 cm, 5 um; Mobile Phase A: Hex:DCM=3:1 w/10 mM NH₃—MeOH, Mobile Phase B: EtOH; Flow rate: 40 mL/min; Gradient: 30% B for 16 min; 220/254 nm. The stereochemistry was assigned based on an x-ray crystal structure of Example 1 Isomer A.
Example 1 Isomer A (1A): 5-(((S)-1-((R)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (549 mg, 21% yield, white solid). LC-MS: (ESI, m z): 540.2 [M+H]⁺; ¹H NMR (300 MHz, DMSO-d₆) δ12.45 (s, 1H), 8.73 (s, 2H), 7.92 (s, 1H), 6.33-6.25 (m, 1H), 5.30 (m, 1H), 4.52-4.42 (m, 1H), 4.12-4.08 (m, 1H), 3.96-3.72(m, 4H), 3.71-3.36 (m, 8H), 1.15 (d, J=6.4 Hz, 3H). Chiral HPLC: CHIRALPAK IG-3 (0.46*5 cm; 3 um); detected at 254 nm; Hex:DCM=3:1 w/ 0.1% DEA:EtOH=80:20; flow rate=1 mL/min; tR=2.85 min.
Example 1 Isomer B (1B): 5-(((S)-1-((S)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (480 mg, 18% yield, white solid). LC-MS: (ESI, m z): 540.2 [M+H]⁺; ¹H NMR (300 MHZ, DMSO-d₆) δ12.46 (s, 1H), 8.73 (s, 2H), 7.91 (s, 1H), 6.34-6.25 (m, 1H), 5.30 (d, J=6.8 Hz, 1H), 4.52-4.42 (m, 1H), 4.11-4.07 (m, 1H), 3.92-3.71 (m, 4H), 3.71-3.40 (m, 8H), 1.15 (d, J=6.4 Hz, 3H). Chiral HPLC: CHIRALPAK IG-3 (0.46*5 cm; 3 um); detected at 254 nm; Hex:DCM=3:1 w/ 0.1% DEA : EtOH=80:20; flow rate=1 mL/min; tR=3.74 min.

Examples 2-4 in the table below were synthesized according to the procedures described for the synthesis of 5-(((S)-1-((R)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one and 5-(((S)-1-((S)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (see Example 1) using appropriate building blocks and modified reaction conditions (such as reagent ratio, temperature, coupling conditions, and reaction time) as needed. The relative stereochemistry of compounds was assigned by analogy to Example 1, based on the PARP7 potency of the more active diastereomer and in analogy to the Example 1A X-ray crystal structure.

| Ex. | | |
|---|---|---|
| No. | Structure | Analytical Data |
| 2A | 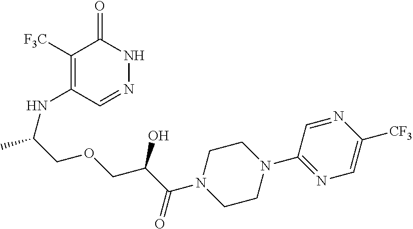 [SMILES: C[C@@H](COC[C@@H](O)C(=O)N1CCN(c2cnc(C(F)(F)F)cn2)CC1)Nc1cn[nH]c(=O)c1C(F)(F)F] | LC-MS: (ESI, m/z): 540.20 [M + H]⁺, ¹H NMR (300 MHz, Methanol-d₄) δ 8.44 (s, 1H), 8.31 (d, J = 0.9 Hz, 1H), 7.96 (s, 1H), 4.65 (t, J = 5.4 Hz, 1H), 4.25-4.15 (m, 1H), 3.83-3.40 (m, 12H), 1.28 (d, J = 6.0 Hz, 3H). Chiral HPLC: CHIRALPAK IC-3, 4.6*50 mm, 3 um; detected at 254 nm; hexane:DCM = 1:1 w/(0.1% isopropylamine)/IPA = 50/50; flow rate = 1 mL/min; rT = 2.454 min |
| | 5-(((S)-1-((R)-2-hydroxy-3-oxo-3-(4-(5- | |
| | (trifluoromethyl)pyrazin-2-yl)piperazin-1- | |
| | yl)propoxy)propan-2-yl)amino)-4- | |
| | (trifluoromethyl)pyridazin-3(2H)-one | |
| 2B | 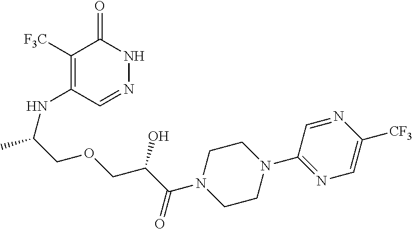 [SMILES: C[C@@H](COC[C@H](O)C(=O)N1CCN(c2cnc(C(F)(F)F)cn2)CC1)Nc1cn[nH]c(=O)c1C(F)(F)F] | LC-MS: (ESI, m/z): 540.2 [M + H]⁺, ¹H NMR (300 MHz, Methanol-d₄) δ 8.43 (s, 1H), 8.31 (d, J = 1.2 Hz, 1H), 7.98 (s, 1H), 4.64 (t, J = 5.4 Hz, 1H), 4.25-4.15 (m, 1H), 3.81-3.50 (m, 12H), 1.28 (d, J = 6.6 Hz, 3H). Chiral HPLC: CHIRALPAK IC-3, 4.6*50 mm, 3 um; detected at 254 nm; hexane:DCM = 1:1 w/(0.1% isopropylamine)/IPA = 50/50; flow rate = 1 mL/min; rT = 3.566 min. |
| | 5-(((S)-1-((S)-2-hydroxy-3-oxo-3-(4-(5- | |
| | (trifluoromethyl)pyrazin-2-yl)piperazin-1- | |
| | yl)propoxy)propan-2-yl)amino)-4- | |
| | (trifluoromethyl)pyridazin-3(2H)-one | |
| 3A | 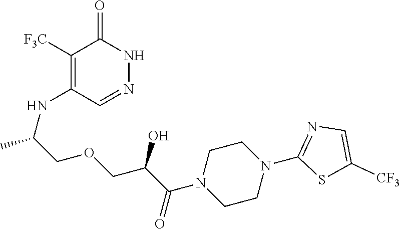 [SMILES: C[C@@H](COC[C@@H](O)C(=O)N1CCN(c2ncc(C(F)(F)F)s2)CC1)Nc1cn[nH]c(=O)c1C(F)(F)F] | LCMS (ESI, m/z): 545.10 [M + H]⁺; ¹H NMR (300 MHz, Methanol-d₄) δ 7.97 (s, 1H), 7.58 (s, 1H), 4.63 (t, J = 5.7 Hz, 1H), 4.18-4.05 (m, 1H), 3.82- 3.55 (m, 12H), 1.28 (d, J = 6.0 Hz, 3H); Chiral HPLC: CHIRALPAK IC-3, 4.6*50 mm, 3 um; detected at 254 nm; Hex:DCM = 5:1 w/0.1% DEA/ EtOH = 50/50; flow = 1 mL/min; rT = 1.657 min. |
| | 5-((S)-1-((R)-2-hydroxy-3-oxo-3-(4-(5- | |
| | (trifluoromethyl)thiazol-2-yl)piperazin-1- | |
| | yl)propoxy)propan-2-ylamino)-4- | |
| | (trifluoromethyl)pyridazin-3(2H)-one | |
| 3B | 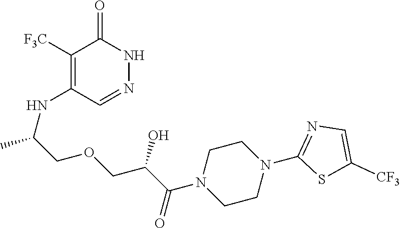 [SMILES: C[C@@H](COC[C@H](O)C(=O)N1CCN(c2ncc(C(F)(F)F)s2)CC1)Nc1cn[nH]c(=O)c1C(F)(F)F] | LC-MS: (ESI, m/z): 545.10 [M + H]⁺; ¹H NMR (300 MHz, Methanol-d₄) δ 7.96 (s, 1H), 7.58 (s, 1H), 4.63 (t, J = 5.4 Hz, 1H), 4.18-4.05 (m, 1H), 3.89- 3.54 (m, 12H), 1.28 (d, J = 6.0 Hz, 3H); Chiral HPLC: CHIRALPAK IC-3, 4.6*50 mm, 3 um; detected at 254 nm; Hex:DCM = 5:1 w/0.1% DEA/ EtOH = 50/50; flow = 1 mL/min; rT = 2.380 min. |
| | 5-((S)-1-((S)-2-hydroxy-3-oxo-3-(4-(5- | |
| | (trifluoromethyl)thiazol-2-yl)piperazin-1- | |
| | yl)propoxy)propan-2-ylamino)-4- | |
| | (trifluoromethyl)pyridazin-3(2H)-one | |
| 4A | 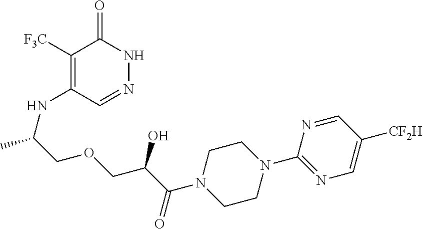 [SMILES: C[C@@H](COC[C@@H](O)C(=O)N1CCN(c2ncc(C(F)F)cn2)CC1)Nc1cn[nH]c(=O)c1C(F)(F)F] | ¹H NMR (300 MHz, DMSO-d₆) δ 12.45 (s, 1H), 8.58 (s, 2H), 7.92 (s, 1H), 6.98 (t, J = 55.6 Hz, 1H), 6.35-6.27 (m, 1H), 5.26 (d, J = 7.2 Hz, 1H), 4.51- 4.41 (m, 1H), 4.22-4.09 (m, 1H), 3.90-3.70 (m, 4H), 3.65- 3.41 (m, 8H), 1.16 (d, J = 6.4 Hz, 3H); LCMS (ESI, m/z): 522.2 [M + H]⁺; Chiral HPLC: CHIRALPAK IG-3, 4.6*50 mm, 3 um; detected at 254 nm; Hex:DCM = 3:1 w/ 0.1% DEA:EtOH = 50:50; Flow |
| | 5-(((S)-1-((R)-3-(4-(5- | rate: 1 mL/min; tR = 2.458 min. |
| | (difluoromethyl)pyrimidin-2-yl)piperazin-1- | |
| | yl)-2-hydroxy-3-oxopropoxy)propan-2- | |
| | yl)amino)-4-(trifluoromethyl)pyridazin-3(2H)- | |
| | one | |
| 4B | 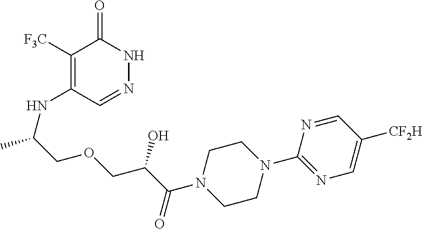 [SMILES: C[C@@H](COC[C@H](O)C(=O)N1CCN(c2ncc(C(F)F)cn2)CC1)Nc1cn[nH]c(=O)c1C(F)(F)F] | ¹H NMR (400 MHz, DMSO-d₆) δ 12.45 (s, 1H), 8.56 (s, 2H), 7.90 (s, 1H), 6.96 (t, J = 55.6 Hz, 1H), 6.35-6.27 (m, 1H), 5.26 (d, J = 7.2 Hz, 1H), 4.51- 4.41 (m, 1H), 4.19-4.09 (m, 1H), 3.90-3.70 (m, 4H), 3.65- 3.42 (m, 8H), 1.13 (d, J = 6.4 Hz, 3H); LCMS (ESI, m/z): 522.2 [M + H]⁺; Chiral HPLC: CHIRALPAK IG-3, 4.6*50 mm, 3 um; detected at 254 nm; Hex:DCM = 3:1 w/ 0.1% DEA:EtOH = 50:50; Flow |
| | 5-(((S)-1-((S)-3-(4-(5- | rate: 1 mL/min; tR = 4.389. |
| | (difluoromethyl)pyrimidin-2-yl)piperazin-1- | |
| | yl)-2-hydroxy-3-oxopropoxy)propan-2- | |
| | yl)amino)-4-(trifluoromethyl)pyridazin-3(2H)- | |
| | one | |

### Example 5: Synthesis of 5-(((2S)-1-(2-hydroxy-2-methyl-3-0x0-3-(4-(5-(trifluoromethyl)pyrimidin-2-yl)piperazin-1-yl)propoxy)propan-2-yl)amino)-4-(trifluoromethyl)pyridazin-3(2H)-one

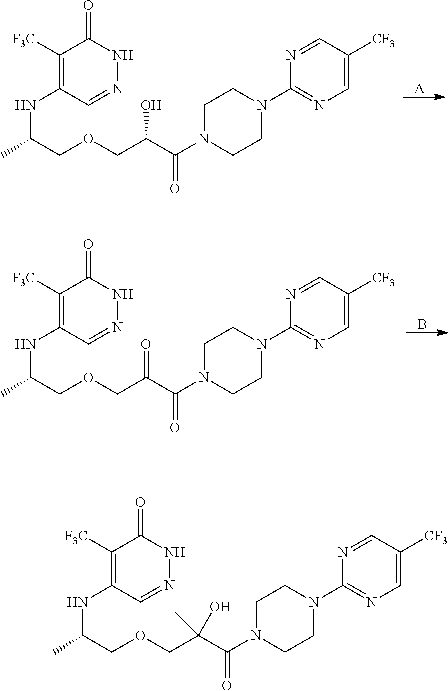

### Step A

To a solution of 5-(((S)-1-((S)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (20 mg, 0.04 mmol, 1.0 equiv) in DCM (2 mL) was added Dess-Martin periodinane (24 mg, 0.06 mmol, 1.5 equiv) in a single portion. After two hours, the solution was poured into 10% aqueous sodium thiosulfate and extracted with dichloromethane (3×20 mL). The organics were washed with saturated sodium bicarbonate, brine, dried over magnesium sulfate, filtered, and the solvent was removed in vacuo. The crude (S)-3-(2-((6-oxo-5-(trifluoromethyl)-1,6-dihydropyridazin-4-yl)amino)propoxy)-1-(4-(5-(trifluoromethyl)pyrimidin-2-yl)piperazin-1-yl)propane-1,2-dione (24 mg) was carried forward without further purification.

### Step B

To a solution of (S)-3-(2-((6-oxo-5-(trifluoromethyl)-1,6-dihydropyridazin-4-yl)amino)propoxy)-1-(4-(5-(trifluoromethyl)pyrimidin-2-yl)piperazin-1-yl)propane-1,2-dione (24 mg, 0.04 mmol, 1 equiv) in THF (1 mL) at −78° C. was added methyl magnesium bromide (0.05 mL, 0.16 mmol, 3.5 equiv) as a 3.0 M solution in diethyl ether over the course of 2 min. After stirring for 15 min, saturated aqueous ammonium chloride was added and the solution was warmed to RT. Additional saturated aqueous ammonium chloride (50 mL) and ethyl acetate (50 mL) was added and the layers were separated. The aqueous layer was extracted with ethyl acetate (3×30 mL). The organics were combined, dried over magnesium sulfate, filtered, and the solvent was removed in vacuo. Purification by silica gel chromatography (0-8% MeOH in DCM) provided 5-(((2S)-1-(2-hydroxy-2-methyl-3-oxo-3-(4-(5-(trifluoromethyl)pyrimidin-2-yl)piperazin-1-yl)propoxy)propan-2-yl)amino)-4-(trifluoromethyl)pyridazin-3(2H)-one (9 mg, 0.016 mmol, 36% yield) as a film. LCMS (ESI, m/z): 554.0 [M+H]⁺; ¹H NMR (300 MHz, Methanol-d₄) δ8.57 (s, 2H), 7.95 (d, J=4 Hz, 1H), 4.17-4.12 (m, 1H), 3.97-3.85 (m, 6H), 3.79 (dd, J=8.0 Hz, 4.0 Hz, 1H), 3.65 (dt, J=8.0, 4.0 Hz, 1H), 3.57-3.51 (m, 2H), 1.40 (s, 3H); 1.26 (d, J=8.0 Hz, 3H).

### Example 6: Synthesis of 5-(((S)-1-((R)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one (6A) and 5-(((S)-1-((S)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one (6B)

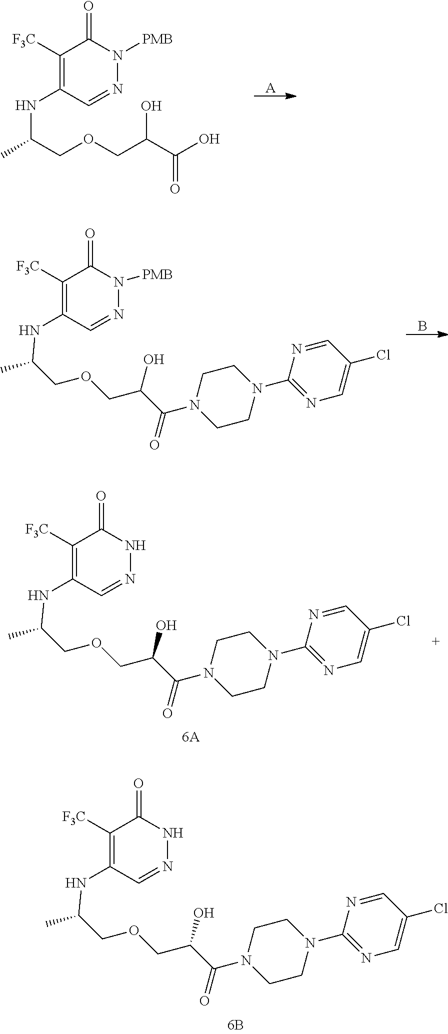

### Step A

A solution of 2-hydroxy-3-((S)-2-((1-(4-methoxy benzyl)-6-oxo-5-(trifluoromethyl)-1,6-dihydropyridazin-4-yl)oxy)propoxy)propanoic acid (200 mg, 0.45 mmol, 1.0 equiv), 5-chloro-2-(piperazin-1-yl)pyrimidine (107 mg, 0.54 mmol, 1.2 equiv), DIEA (174 mg, 1.35 mmol, 3.0 equiv), and HATU (256 mg, 0.67 mmol, 1.50 equiv) in DMF (2.0 mL) was stirred for 1 hour at room temperature. The crude product was purified by reverse phase column chromatography to afford 75 mg (27% yield) of 5-(((2S)-1-(3-(4-(5-chloropyrimidin-2-yl)piperazin-1-yl)-2-hydroxy-3-oxopropoxy)propan-2-yl)amino)-2-(4-methoxy benzyl)-4-(trifluoromethyl)pyridazin-3(2H)-one as a yellow oil. (ES, m/z): 626.20 [M+H]⁺.

### Step B

A solution of 5-(((2S)-1-(3-(4-(5-chloropyrimidin-2-yl)piperazin-1-yl)-2-hydroxy-3-oxopropoxy)propan-2-yl)amino)-2-(4-methoxy benzyl)-4-(trifluoromethyl)pyridazin-3(2H)-one (75 mg. 0.12 mmol. 1.0 equiv) in TFA (3.0 mL) and triflic acid (0.3 mL) was stirred for 1 h at −10° C. Ice water (5 mL) was added and the pH value of the solution was adjusted to 8 with saturated NaHCO; aqueous solution. The resulting solution was extracted with 3×10 mL of ethyl acetate. The organic layers were combined. dried over anhydrous sodium sulfate and concentrated under vacuum. The crude product was purified by reverse phase prep-HPLC to afford 50 mg of a diastereomeric mixture. The product was further purified by chiral HPLC with the following conditions (CHIRALPAK IG. 2.0*25 cm, 5 um; mobile phase A: Hex:DCM=3:1 w/ 10 mM NH₃-MeOH, mobile phase B: EtOH; Flow rate: 20 mL/min; Gradient: 50% B for 14 min) to afford the desired isomers. The relative stereochemistry of compounds was assigned by analogy to Example 1. based on the PARP7 potency of the more active diastereomer and in analogy to the Example 1A X-ray crystal structure.
Example 6 Isomer A (6A): 5-(((S)-1-((R)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one (23 mg, 46% yield) as a white solid. LCMS (ESI, m/z): 506.10 [M+H]⁺; ¹H NMR (300 MHZ, DMSO-d₆) δ12.45 (s, 1H), 8.45 (s, 2H), 7.92 (s, 1H), 6.30-6.21 (m, 1H), 5.27 (d, J=7.2 Hz, 1H), 4.48 (d, J=6.5 Hz, 1H), 4.21-4.15 (m. 1H). 3.80-3.49 (m, 12H), 1.15 (d, J=6.4 Hz, 3H) Chiral HPLC: CHIRALPAK IC-3. 4.6*50mm 3 um; Hex:DCM=3:1 w/0.1% DEA:EtOH=50:50; Flow rate: 1 mL/min; tR=2.488 min.
Example 6 Isomer B (6B): 5-(((S)-1-((S)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one (24 mg, 48% yield) as a white solid. LCMS (ESI, m/z): 506.10 [M+H]⁺; ¹H NMR (300 MHZ, DMSO-d₆) δ12.46 (s, 1H), 8.45 (s, 2H), 7.91 (s, 1H), 6.30-6.21 (m, 1H), 5.27 (d, J=7.2 Hz. 1H), 4.48 (d. J=6.7 Hz. 1H), 4.19-4.13 (m, 1H), 3.80-3.49 (m, 12H), 1.14 (d, J=6.4 Hz, 3H), Chiral HPLC: CHIRALPAK IC-3. 4.6*50 mm 3 um; Hex:DCM=3:1 w/ 0.1% DEA:EtOH=50:50; Flow rate: 1 mL/min; tR=3.444 min.

### Example 7: Synthesis of 5-(((S)-1-((R)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one (7A) and 5-(((S)-1-((S)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one (7B)

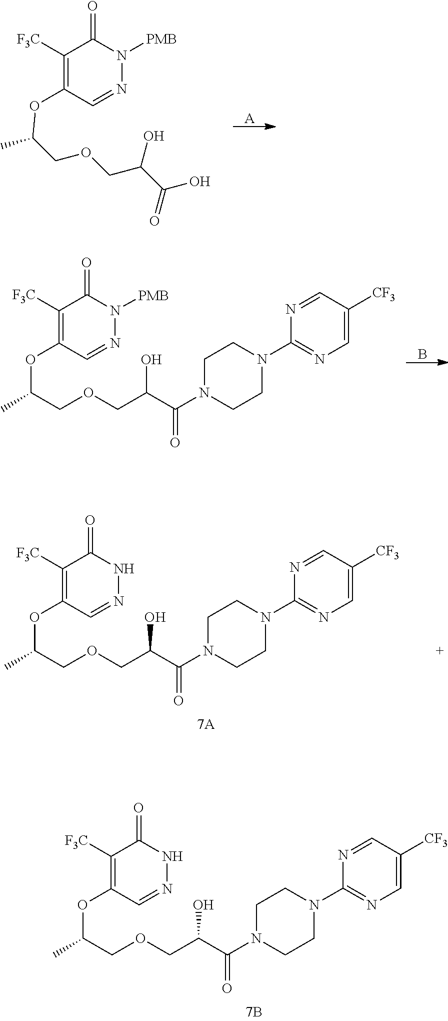

### Step A

A solution of 2-hydroxy-3-((S)-2-((1-(4-methoxy benzyl)-6-oxo-5-(trifluoromethyl)-1,6-dihydropyridazin-4-yl)oxy)propoxy)propanoic acid (315 mg, 0.71 mmol, 1.0 equiv), DIEA (182 mg, 1.41 mmol, 2.0 equiv), HATU (215 mg, 0.57 mmol, 0.80 equiv) and 2-(piperazin-1-yl)-5-(trifluoromethyl)pyrimidine (131 mg, 0.57 mmol, 0.80 equiv) in DMF (5 mL) was stirred for 1 hour. The solution was applied onto a reverse phase column eluting with H₂O/CH₃CN (28/72) to afford 5-(((2S)-1-(2-hydroxy-3-oxo-3-(4-(5-(trifluoromethyl)pyrimidin-2-yl)piperazin-1-yl)propoxy)propan-2-yl)oxy)-2-(4-methoxy benzyl)-4-(trifluoromethyl)pyridazin-3(2H)-one (257 mg, 45% yield) as a yellow oil. LCMS (ESI, m/z): 661.20 [M+H]⁺.

### Step B

To a solution of 5-(((2S)-1-(2-hydroxy-3-oxo-3-(4-(5-(trifluoromethyl)pyrimidin-2-yl)piperazin-1-yl)propoxy)propan-2-yl)oxy)-2-(4-methoxy benzyl)-4-(trifluoromethyl)pyridazin-3(2H)-one (100 mg, 0.15 mmol, 1.0 equiv) in TFA (0.70 mL) at 0° C. was added triflic acid (113 mg, 0.76 mmol, 5.0 equiv) and the resulting solution was stirred for 1 hour at that temperature. Ice water (50 mL) was added and the pH value of the solution was adjusted to 7 with saturated aqueous NaHCO₃. The resulting solution was extracted with 3×200 mL of dichloromethane. The organic layers were combined, dried over anhydrous sodium sulfate and concentrated under vacuum. The residue was applied onto a reverse phase column eluting with H₂O/CH₃CN (42/58) to afford the product as a mixture of diastereomers. The product was further purified by chiral-prep-HPLC with following conditions: (CHIRALPAK IA, 2*25 cm, 5 um; Mobile Phase A: Hex w/ 8mM NH₃—MeOH, Mobile Phase B: EtOH; Flow rate: 16 mL/min; Gradient: 50% B for 20 min; 254/220 nm) to afford the separated isomers. The relative stereochemistry of compounds was assigned by analogy to Example 1, based on the PARP7 potency of the more active diastereomer and in analogy to the Example 1A X-ray crystal structure.
Example 7 Isomer A (7A): 5-(((S)-1-((R)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one (10 mg, 11%, white solid). LC-MS: (ESI, m/z): 541.20 [M+H]⁺; ¹H NMR (300 MHz, Methanol-d₄) δ8.61 (s, 2H), 8.26 (s, 1H), 5.21-5.12 (m, 1H), 4.60 (t, J=7.5 Hz, 1H), 4.10-3.87 (m, 4H), 3.79-3.64 (m, 8H), 1.37 (d, J=5.4 Hz, 3H). Chiral HPLC: CHIRALPAK IA-3, 4.6*50 mm, 3 um; detected at 254 nm; Hex w/0.1% DEA/EtOH=50/50; flow rate=1 mL/min; tR=1.749 min.
Example 7 Isomer B (7B): 5-(((S)-1-((S)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one (7 mg, 8% yield, white solid). LC-MS: (ESI, m/z): 541.20 [M+H]⁺; ¹H NMR (300 MHz, Methanol-d₄) δ8.61 (s, 2H), 8.23 (s, 1H), 5.21-5.12 (m, 1H), 4.62 (t, J=5.4 Hz, 1H), 4.10-3.87 (m, 4H), 3.79-3.64 (m, 8H), 1.37 (d, J=6.0 Hz, 3H). Chiral HPLC: CHIRALPAK IA-3, 4.6*50 mm, 3 um; detected at 254 nm; Hex w/0.1% DEA/EtOH=50/50; flow rate=1 mL/min; tR=4.163min.

Example 8 was synthesized according to the procedures described for the synthesis of 5-(((S)-1-((R)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one and 5-(((S)-1 -((S)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one (see Example 7) using appropriate building blocks and modified reaction conditions (such as reagent ratio, temperature, coupling conditions, and reaction time) as needed.

| Ex. | | |
|---|---|---|
| No. | Structure | Analytical Data |
| 8A | 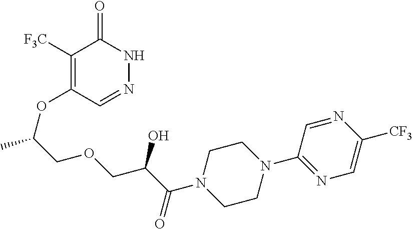 [SMILES: C[C@@H](COC[C@@H](O)C(=O)N1CCN(c2cnc(C(F)(F)F)cn2)CC1)Oc1cn[nH]c(=O)c1C(F)(F)F] | LC-MS: (ESI, m/z): 541.10 [M + H]⁺; ¹H NMR (300 MHz, Methanol-d₄) δ 8.43 (s, 1H), 8.32 (s, 1H), 8.31 (s, 1H), 5.21- 5.15 (m, 1H), 4.59 (t, J = 5.7 Hz, 1H), 3.85-3.56 (m, 12H), 1.37 (d, J = 6.0 Hz, 3H). Chiral HPLC: CHIRALPAK IF-3, 4.6*50 mm; 3 um; detected at 254 nm; Hex w/0.1% DEA/ EtOH = 50/50; flow = 1 mL/min; rT = 1.833 min. |
| | 5-(((S)-1-((R)-2-hydroxy-3-oxo-3-(4-(5- | |
| | (trifluoromethyl)pyrazin-2-yl)piperazin-1- | |
| | yl)propoxy)propan-2-yl)oxy)-4- | |
| | (trifluoromethyl)pyridazin-3(2H)-one | |
| 8B | 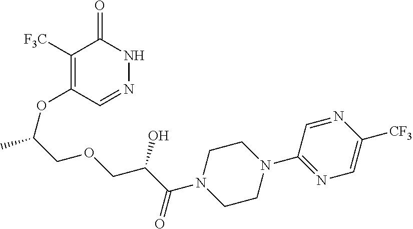 [SMILES: C[C@@H](COC[C@H](O)C(=O)N1CCN(c2cnc(C(F)(F)F)cn2)CC1)Oc1cn[nH]c(=O)c1C(F)(F)F] | LC-MS: (ESI, m/z): 541.20 [M + H]⁺; ¹H NMR (300 MHz, Methanol-d₄) δ 8.43 (s, 1H), 8.31 (s, 1H), 8.23 (s, 1H), 5.20-5.14 (m, 1H), 4.61 (t, J = 5.4 Hz, 1H), 3.84- 3.56 (m, 3H), 1.37 (d, J = 6.3 Hz, 3H). Chiral HPLC: CHIRALPAK IF-3, 4.6*50 mm; 3 um; detected at 254 nm; Hex w/0.1% DEA/ EtOH = 50/50; flow = 1 mL/min; rT = 2.502 min. |
| | 5-(((S)-1-((S)-2-hydroxy-3-oxo-3-(4-(5- | |
| | (trifluoromethyl)pyrazin-2-yl)piperazin-1- | |
| | yl)propoxy)propan-2-yl)oxy)-4- | |
| | (trifluoromethyl)pyridazin-3(2H)-one | |

### Example 9: Synthesis of 5-(((S)-1-((R)-3-(4-(5-chloropyrimidin-2-yl)piperazin-1-yl)-2-hydroxy-3-oxopropoxy)propan-2-yl)oxy)-4-(trifluoromethyl)pyridazin-3(2H)-one

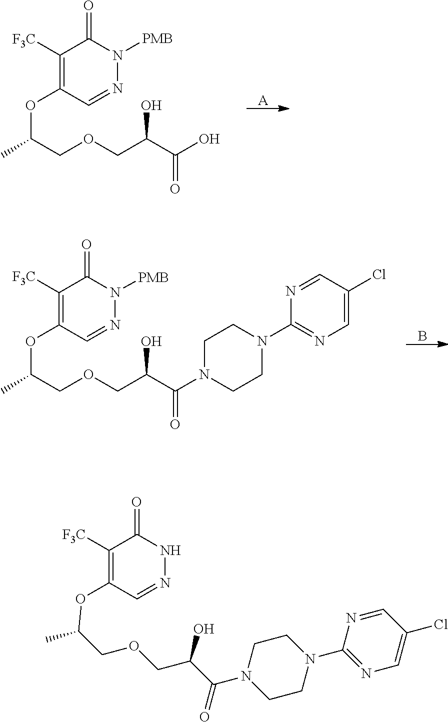

### Step A

A solution of (R)-2-hydroxy-3-((S)-2-((1-(4-methoxy benzyl)-6-oxo-5-(trifluoromethyl)-1,6-dihydropyridazin-4-yl)oxy)propoxy)propanoic acid (100 mg, 0.224 mmol, 1.00 equiv), 5-chloro-2-(piperazin-1-yl)pyrimidine hydrochloride (58 mg, 0.246 mmol, 1.10 equiv), HATU (102.22 mg, 0.269 mmol, 1.20 equiv), and DIEA (86 mg, 0.672 mmol, 3.00 equiv) in DMF (2 mL) was stirred for 2 hours. 10 mL of water was added and the resulting solution was extracted with 3×15 mL of ethyl acetate. The organic layers were combined, dried over anhydrous sodium sulfate and concentrated. The crude product was purified by reverse phase chromatography eluting with MeCN/H₂O (70/30) to afford 50 mg (36% yield) of 5-(((S)-1-((R)-3-(4-(5-chloropyrimidin-2-yl)piperazin-1-yl)-2-hydroxy-3-oxopropoxy)propan-2-yl)oxy)-2-(4-methoxy benzyl)-4-(trifluoromethyl)pyridazin-3(2H)-one as a white solid. LCMS (ESI, m/z): 627.20 [M+H]⁺.

### Step B

A solution of 5-(((S)-1-((R)-3-(4-(5-chloropyrimidin-2-yl)piperazin-1-yl)-2-hydroxy-3-oxopropoxy)propan-2-yl)oxy)-2-(4-methoxy benzyl)-4-(trifluoromethyl)pyridazin-3(2H)-one (40 mg, 0.064 mmol, 1.0 equiv) in 3 mL of a 10:1 mixture of trifluoroacetic acid and trifluoromethanesulfonic acid was stirred for 1 hour at 0° C. 10 mL of ice water was added and the pH value of the solution was adjusted to 5-6 with saturated aqueous Na₂CO₃. The resulting solution was extracted with 3×10 mL of ethyl acetate. The organic layers were combined and concentrated. The crude product was purified by reverse phase chromatography eluting with H₂O/CH₃CN (60/40). The product was further purified by chiral prep-HPLC: (CHIRALPAK IA, 2*25 cm, 5 um; Mobile Phase A: Hex:EtOH=1:1 w/8 mM NH₃—MeOH); Mobile Phase B: EtOH; 50% B for 16 minutes; 220/254 nm) to afford the desired product. LCMS (ESI, m/z): 507.15 [M+H]⁺; ¹H NMR (400 MHZ, Methanol-d₄) δ8.33 (s, 2H), 8.24 (s, 1H), 5.10-5.20 (m, 1H), 4.60 (t, J=5.6 Hz, 1H), 3.90-3.78 (m, 4H), 3.76-3.64 (m, 7H), 3.62-3.58 (m, 2H), 1.36 (d, J=6.3 Hz, 3H). Chiral HPLC: (CHIRALPAK IA-3, 4.6*50 mm 3 um, Hex w/0.1%DEA : EtOH=50:50; Flow rate: 1 mL/min: rT=2.360 min.)

### Example 10: Synthesis of 6-(4-((R)-2-hydroxy-3-((S)-2-((6-oxo-5-(trifluoromethyl)-1,6-dihydropyridazin-4-yl)oxy)propoxy)propanoyl)piperazin-1-yl)nicotinonitrile

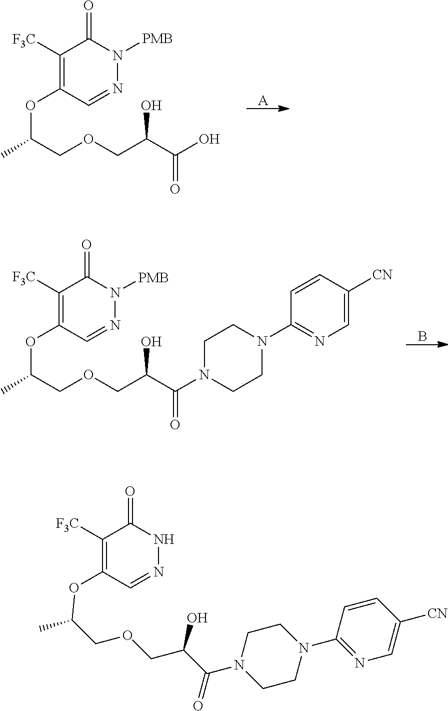

### Step A

A solution of (R)-2-hydroxy-3-((S)-2-((1-(4-methoxy benzyl)-6-oxo-5-(trifluoromethyl)-1,6-dihydropyridazin-4-yl)oxy)propoxy)propanoic acid (120 mg, 0.269 mmol, 1.00 equiv), 6-(piperazin-1-yl)nicotinonitrile (40 mg, 0.215 mmol, 0.80 equiv), DIEA (69 mg, 0.538 mmol, 2.0 equiv) and HATU (82 mg, 0.215 mmol, 0.80 equiv) in DMF (1.00 mL) was stirred for 1 hour. The solution was purified directly by reverse phase chromatography eluting with H₂O/CH₃CN (39/61) to afford 77 mg of 6-(4-((R)-2-hydroxy-3-((S)-2-((1-(4-methoxy benzyl)-6-oxo-5-(trifluoromethyl)-1,6-dihydropyridazin-4-yl)oxy)propoxy)propanoyl)piperazin-1-yl)nicotinonitrile as a crude yellow oil. LCMS (ESI, m/z): 617.20 [M+H]⁺.

### Step B

A solution of 6-(4-((R)-2-hydroxy-3-((S)-2-((1-(4-methoxy benzyl)-6-oxo-5-(trifluoromethyl)-1,6-dihydropyridazin-4-yl)oxy)propoxy)propanoyl)piperazin-1-yl)nicotinonitrile (72 mg, 0.076 mmol, 1.00 equiv) in TFA (1.0 mL) and triflic acid (57 mg, 0.380 mmol, 5.0 equiv) was stirred for 1 hour at 0° C. 20 mL of water was added and the pH value of the solution was adjusted to pH 7 with saturated aqueous NaHCO₃. The resulting solution was extracted with 3×100 mL of dichloromethane. The organic layers were combined, dried over anhydrous sodium sulfate and concentrated under vacuum. The residue was purified by reverse phase chromatography eluting with H₂O/CH₃CN (50/50). The product was further purified by chiral prep-HPLC with following conditions: (CHIRALPAK IG, 20*250 mm, 5 μm: Mobile Phase A: Hex:DCM=3:1 w/10mM NH₃-MeOH, Mobile Phase B: EtOH: Flow rate: 18 mL/min; 50% B for 15 min; 254/220 nm) to afford the desired product (3.1 mg, 8% yield) as a white solid. LC-MS: (ESI, m/z): 497.10 [M+H]⁺; ¹H NMR (300 MHZ, Methanol-d₄) δ8.44 (s, 1H), 8.26 (s, 1H), 7.78 (d, J=9.3 Hz, 1H), 6.87 (d, J=9.0 Hz, 1H), 5.21-5.21 (m, 1H), 4.60 (t, J=6.0 Hz, 1H), 3.85-3.56 (m, 12H), 1.36 (d, J=6.6 Hz, 3H). Chiral HPLC: CHIRALPAK IG-3, 4.6*50 mm; 3 μm; detected at 254 nm; Hex:DCM=3:1 w/0.1% DEA/EtOH=50/50; flow=1 mL/min; rT=1.951 min.

### Example 11: Synthesis of 5-(((S)-1-((R)-3-(4-(5-(difluoromethyl)pyrimidin-2-yl)piperazin-1-yl)-2-hydroxy-3-oxopropoxy)propan-2-yl)oxy)-4-(trifluoromethyl)pyridazin-3(2H)-one

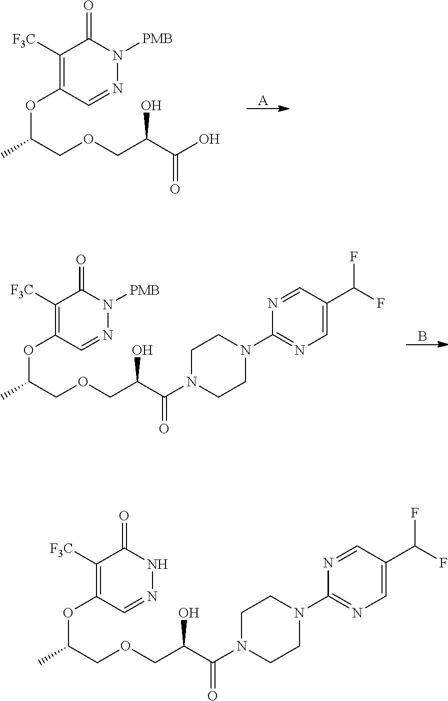

### Step A

A solution of (R)-2-hydroxy-3-((S)-2-((1-(4-methoxybenzyl)-6-oxo-5-(trifluoromethyl)-1,6-dihydropyridazin-4-yl)oxy)propoxy)propanoic acid (60 mg, 0.134 mmol, 1.00 equiv), 5-(difluoromethyl)-2-(piperazin-1-yl)pyrimidine (29 mg, 0.134 mmol, 1.00 equiv), HATU (61 mg, 0.161 mmol, 1.20 equiv), and DIEA (52 mg, 0.403 mmol, 3.00 equiv) in DMF (2.00 mL) was stirred for 2 hours at room temperature. 10 mL of water was added. The resulting solution was extracted with 3×15 mL of ethyl acetate. The organic layers were combined, dried, and concentrated. The crude product was applied onto a reverse phase column eluting with H₂O/CH₃CN (30/70) to afford 36 mg (42% yield) of 5-(((S)-1-((R)-3-(4-(5-(difluoromethyl)pyrimidin-2-yl)piperazin-1-yl)-2-hydroxy-3-oxopropoxy)propan-2-yl)oxy)-2-(4-methoxy benzyl)-4-(trifluoromethyl)pyridazin-3(2H)-one as a white solid. LCMS: [M+H]⁺643.20.

### Step B

A solution of 5-(((S)-1-((R)-3-(4-(5-(difluoromethyl)pyrimidin-2-yl)piperazin-1-yl)-2-hydroxy-3-oxopropoxy)propan-2-yl)oxy)-2-(4-methoxy benzyl)-4-(trifluoromethyl)pyridazin-3(2H)-one (36 mg, 0.056 mmol, 1 equiv) in 3 mL of TFA/trifluoromethanesulfonic acid (10/1) was stirred for 1 hour at 0° C. 10 mL of ice water was added and the pH of the solution was adjusted to 5˜6 with saturated aqueous Na₂CO₃. The resulting solution was extracted with 3×10 mL of ethyl acetate. The organic layers were combined, dried over anhydrous sodium sulfate and concentrated. The crude product was purified by reverse phase chromatography eluting with H₂O/CH₃CN (55/45). The product was further purified by chiral prep-HPLC with the following conditions: (CHIRALPAK IG, 2.0 cm*25 cm, 5 μm; mobile phase A Hex:DCM=3:1 w/10 mM NH₃-MEOH, mobile phase B EtOH; Flow rate: 18 mL/min; Gradient: 50% EtOH for 18 min; 220/254 nm) to afford 5-(((S)-1-((R)-3-(4-(5-(difluoromethyl)pyrimidin-2-yl)piperazin-1-yl)-2-hydroxy-3-oxopropoxy)propan-2-yl)oxy)-4-(trifluoromethyl)pyridazin-3(2H)-one (5.3 mg, 18% yield) as a whites solid. LCMS (ESI, m/z): 523.15 [M+H]⁺; ¹H-NMR (400 MHZ, Methanol-d₄) δ8.50 (s, 2H), 8.25 (s, 1H), 6.75 (t, J=52 Hz 1H), 5.21-5.10 (m, 1H), 4.60 (t, J=5.5 Hz, 1H), 4.02-3.80 (m, 4H), 3.78-3.62 (m, 8H), 1.36 (d, J=6.4 Hz, 3H). Chiral HPLC: CHIRALPAK IG-3, 3.0*50 mm, 3 μm; Hex:DCM=3:1 w/8 mM NH₃-MeOH:EtOH=50:50, Flow rate: 1 mL/min; rT=1.724 min.

### Example 12: Synthesis of 4-bromo-5-(((S)-1-((R)-2-hydroxy-3-oxo-3-(4-(5-(trifluoromethyl)pyrimidin-2-yl)piperazin-1-yl)propoxy)propan-2-yl)oxy)pyridazin-3(2H)-one (12A) and 4-bromo-5-(((S)-1-((S)-2-hydroxy-3-oxo-3-(4-(5-(trifluoromethyl)pyrimidin-2-yl)piperazin-1-yl)propoxy)propan-2-yl)oxy)pyridazin-3(2H)-one (12B)

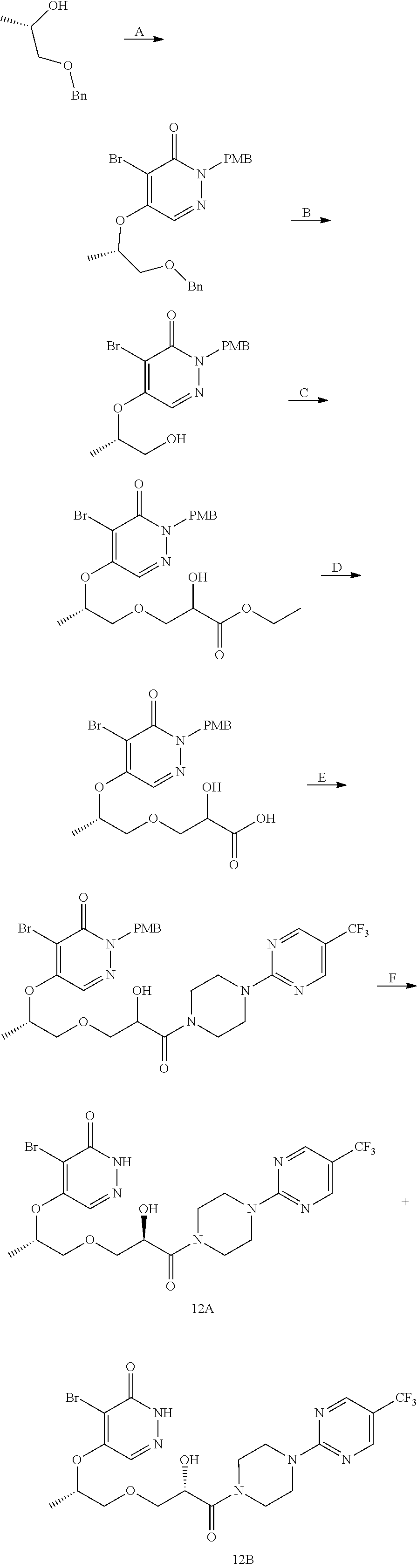

Step A

A solution of 4,5-dibromo-2-(4-methoxybenzyl)pyridazin-3(2H)-one (7.50 g, 20.1 mmol, 1.0 equiv), (S)-1-(benzyloxy)propan-2-ol (5.00 g, 30.08 mmol, 1.5 equiv), and Cs₂CO₃ (9.80 g, 30.08 mmol, 1.5 equiv) in CH₃CN (100 mL) was stirred for 35 hours at 80° C. After filtration, the filtrate was concentrated under vacuum and the crude product was applied onto a silica gel column eluting with ethyl acetate/petroleum ether (55:45) to afford 3.4 g (37% yield) of (S)-5-((1-(benzyloxy)propan-2-yl)oxy)-4-bro mo-2-(4-methoxybenzyl)pyridazin-3(2H)-one as an oil. LCMS (ESI, m/z): 459.00 [M+H]⁺.

### Step B

To a solution of (S)-5-((1-(benzyloxy)propan-2-yl)oxy)-4-bromo-2-(4-methoxybenzyl)pyridazin-3(2H)-one (3.4 g, 7.4 mmol, 1.0 equiv) in DCM (35 mL) was added BCl₃ (1M in DCM, 14.8 mL, 2.0 equiv) at 0° C. and the resulting solution was stirred for 2 hours at room temperature. The reaction was then quenched by the addition of ice water. The resulting solution was extracted with 100 mL of dichloromethane and the organic layers were combined, dried over anhydrous sodium sulfate and concentrated under vacuum. The crude product was applied onto a silica gel column eluting with ethyl acetate/petroleum ether (2:3) to afford 1.1g (52% yield) of (S)-4-bromo-5-((1-hydroxypropan-2-yl)oxy)-2-(4-methoxybenzyl)pyridazin-3(2H)-one as a brown oil. LCMS (ESI, m/z): 369.00 [M+H]⁺.

### Step C

A solution of (S)-4-bromo-5-((1-hydroxypropan-2-yl)oxy)-2-(4-methoxybenzyl)pyridazin-3(2H)-one (700 mg, 1.90 mmol, 1.0 equiv), ethyl oxirane-2-carboxylate (968 mg, 9.48 mmol, 5.0 equiv) and Mg(ClO₄)₂ (306 mg, 0.95 mmol, 0.50 equiv) in ethyl acetate (16 mL) was stirred for 80 hours at 80° C. The solution was filtered and the filtrate was concentrated. The crude product was applied onto a reverse phase column eluting with H₂O:CH₃CN (48/52) to afford 160 mg (18% yield) of ethyl 3-((S)-2-((5-bromo-1-(4-methoxybenzyl)-6-oxo-1,6-dihydropyridazin-4-yl)oxy)propoxy)-2-hydroxypropanoate as an oil. LCMS (ESI, m/z): 487.10[M+H]⁺.

### Step D

To a solution of ethyl 3-((S)-2-((5-bromo-1-(4-methoxy benzyl)-6-oxo-1,6-dihydropyridazin-4-yl)oxy)propoxy)-2-hydroxypropanoate (140 mg, 0.29 mmol, 1.00 equiv) in EtOH (3.00 mL) at 0° C. was added a solution of LiOH.H₂O (28 mg, 0.67 mmol, 2.3 equiv) in H₂O (0.5 mL). The resulting solution was stirred for 1 hour at 0° C., concentrated under vacuum, and applied onto a reverse phase column eluting with H₂O:CH₃CN (25:75) to afford 3-((S)-2-((5-bromo-1-(4-methoxybenzyl)-6-oxo-1,6-dihydropyridazin-4-yl)oxy)propoxy)-2-hydroxypropanoic acid as an oil. LCMS (ESI, m/z): 459.00 [M+H]⁺.

### Step E

A solution of 3-((S)-2-((5-bromo-1-(4-methoxybenzyl)-6-oxo-1,6-dihydropyridazin-4-yl)oxy)propoxy)-2-hydroxypropanoic acid (90 mg, 0.20 mmol, 1.0 equiv), HATU (112 mg, 0.30 mmol, 1.50 equiv), DIEA (51 mg, 0.40 mmol, 2.0 equiv), and 2-(piperazin-1-yl)-5-(trifluoromethyl)pyrimidine (69 mg, 0.30 mmol, 1.50 equiv) in DMF (2 mL) was stirred for 2 hours at room temperature. 20 ml of water was added and ethyl acetate (15 mL) was added. The layers were separated and the aqueous layer was extracted with EtOAc (2×15 mL). The organic layers were combined, washed with 3×15 mL of brine, dried over anhydrous sodium sulfate and concentrated under vacuum. The crude product was applied onto a silica gel column eluting with MeOH/DCM (1:1) to afford 120 mg (91% yield) of 4-bromo-5-(((2S)-1-(2-hydroxy-3-oxo-3-(4-(5-(trifluoromethyl)pyrimidin-2-yl)piperazin-1-yl)propoxy)propan-2-yl)oxy)-2-(4-methoxy benzyl)pyridazin-3(2H)-one as a brown oil. LCMS (ESI, m/z): 671.1 [M+H]⁺.

### Step F

A solution of 4-bromo-5-(((2S)-1-(2-hydroxy-3-oxo-3-(4-(5-(trifluoromethyl)pyrimidin-2-yl)piperazin-1-yl)propoxy)propan-2-yl)oxy)-2-(4-methoxybenzyl)pyridazin-3(2H)-one (100 mg. 0.15 mmol. 1.0 equiv) in trifluoroacetic acid (1 mL) and triflic acid (0).2 ml) was stirred for 1 h at room temperature. Water (5 ml) was added and the solvent was removed in vacuo. After concentration. the crude product was purified by C18 reverse phase column eluting with H₂O/CH₃CN to give a mixture of diastereomers. The product was further purified by chiral prep-HPLC with the following conditions: (CHIRALPAK IF, 2*25 cm, 5 um; Mobile Phase A Hex:DCM=3:1 w/10 mM NH₃-MEOH), Mobile Phase B EtOH; Flow rate: 16 mL/min; Gradient: 50% B for 38 min; 254/220 nm). The relative stereochemistry of the compounds was assigned by analogy to Example 1. based on the PARP7 potency of the more active diastereomer and in analogy to the Example 1A X-ray crystal structure.
Example 12 Isomer A (12A): 4-bromo-5-(((S)-1-((R)-2-hydroxy-3-oxo-3-(4-(5-(trifluoromethyl)pyrimidin-2-yl)piperazin-1-yl)propoxy)propan-2-yl)oxy)pyridazin-3(2H)-one (14 mg, 17% yield, yellow solid). LCMS (ESI, m/z): 553.10 [M+H]⁺. ¹HNMR (DMSO-d₆, 300 MHz) δ13.12 (s, 1H), 8.70 (s, 2H), 8.09 (s, 1H), 5.26 (d, J=7.3 Hz, 1H), 5.06-4.95 (m, 1H), 4.48-4.38 (m, 1H), 3.95-3.75 (m, 4H), 3.68-3.45 (m, 8H). 1.24 (d, J=6.2 Hz, 3H). Chiral HPLC: CHIRALPAK IF-3. 0.46*5 cm; 3 um, Hex:DCM=3:1 w/ 0.1% DEA:EtOH=50:50, Flow rate=1.0mL/min; tR=1.641 min.
Example 12 Isomer B (12B): 4-bromo-5-(((S)-1-((S)-2-hydroxy-3-oxo-3-(4-(5-(trifluoromethyl)pyrimidin-2-yl)piperazin-1-yl)propoxy)propan-2-yl)oxy)pyridazin-3(2H)-one (12 mg, 15% yield, yellow solid). LCMS (ESI, m/z): 553.10 [M+H]⁺. ¹HNMR (DMSO-d₆, 300 MHz) δ13.13 (s, 1H), 8.71 (s, 2H), 8.07 (s, 1H), 5.26 (d, J=7.3 Hz, 1H), 5.05-4.93 (m, 1H), 4.51-4.48 (m, 1H), 3.95-3.75 (m, 4H), 3.68-3.45 (m, 8H), 1.24 (d, J=6.2 Hz, 3H). Chiral HPLC: CHIRALPAK IF-3, 0.46*5 cm; 3 um, Hex:DCM=3:1 w/ 0.1% DEA:EtOH=50:50, Flow rate;32 1.0mL/min; tR=2.856 min.

### Example 13: Synthesis of 5-(((S)-1-((R)-2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-4-(trifluoromethyl)pyridazin-3(2H)-one (13A) and 5-(((S)-1-((S)-2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-4-(trifluoromethyl)pyridazin-3(2H)-one (13B)

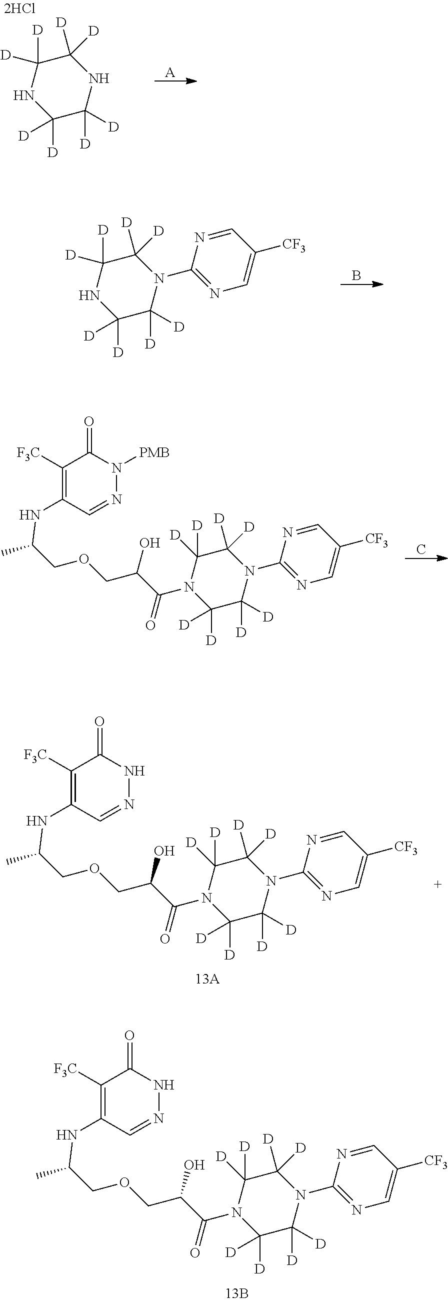

### Step A

To the solution of piperazine-2,2,3,3,5,5,6,6-ds dihydrochloride (2.00 g, 11.97 mmol, 1.0 equiv) and DIEA (3.09 g, 23.94 mmol, 2.0 equiv) in i-PrOH (40 mL) at 0° C. was added 2-chloro-5-(trifluoromethyl)pyrimidine (2.18 g, 11.94 mmol, 1.00 equiv) in i-PrOH (20 mL). The resulting solution was stirred for 1 hour at 0° C. The resulting mixture was concentrated and the crude product was purified by reverse phase chromatography to give two sets of fractions that afforded 1.2 g (66% purity) and 460 mg (81% purity) of 2-(piperazin-1-yl-2,2,3,3,5,5,6,6-d8)-5-(trifluoromethyl)pyrimidine as a white solid. LCMS (ESI, m/z): 241.15 [M+H]⁺.

### Step B

A solution of 2-hydroxy-3-((S)-2-((1-(4-methoxy benzyl)-6-oxo-5-(trifluoromethyl)-1,6-dihydropyridazin-4-yl)amino)propoxy)propanoic acid (770 mg, 1.73 mmol, 1.0 equiv), 2-(piperazin-1-yl-2,2,3,3,5,5,6,6-d₈)-5-(trifluoromethyl)pyrimidine (457 mg, 1.90 mmol, 1.1 equiv), HATU (789 mg, 2.08 mmol, 1.2 equiv), and DIEA (670 mg, 5.19 mmol, 3.0 equiv) in DMF (10 mL) was stirred for 1 hour at room temperature. Ice water (30 mL) was added and the resulting solution was extracted with 3×20 mL of ethyl acetate. The organic layers were combined, dried over magnesium sulfate, filtered, and concentrated in vacuo. The crude product was purified by silica gel chromatography eluting with ethyl acetate to afford 730 mg (63% yield) of 5-(((2S)-1-(2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-2-(4-methoxybenzyl)-4-(trifluoromethyl)pyridazin-3(2H)-one as a yellow oil. LCMS (ESI, m/z): 668.30 [M+H]⁺.

### Step C

A solution of 5-(((2S)-1-(2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-2-(4-methoxy benzyl)-4-(trifluoromethyl)pyridazin-3(2H)-one (730 mg, 1.09 mmol, 1.0 equiv) in TFA (5 ml) and trifluoromethanesulfonic acid (0.50 mL) was stirred for 1 hour at room temperature. Ice water (20 mL) was added and the pH was adjusted to 6 with saturated aqueous sodium carbonate solution. The resulting solution was extracted with 3×20 mL of dichloromethane. The organic layers were recombined, dried over anhydrous sodium sulfate and concentrated under vacuum. The crude product was purified by reverse phase chromatography to afford a mixture of diastereomers. The product was further purified by chiral prep-HPLC with the following conditions: (CHIRALPAK IG, 3*25 cm, 5 μm; mobile phase A: 3:1 Hex:DCM w/10 mM NH₃-MEOH, mobile phase B: EtOH; flow rate: 16 mL/min, Gradient 30% B for 16 min; 220/254 nm) to afford the separated isomers. The relative stereochemistry of the compounds was assigned by analogy to Example 1, based on the PARP7 potency of the more active diastereomer and in analogy to the Example 1A X-ray crystal structure.
Example 13 Isomer A (13A): 5-(((S)-1-((R)-2-hydroxy-3-oxo-3-(4-(5-(trifluoromethyl)pyrimidin-2-yl)piperazin-1-yl-2,2,3,3,5,5,6,6-d₈)propoxy)propan-2-yl)amino)-4-(trifluoromethyl)pyridazin-3(2H)-one (131 mg, 22% yield, white solid). LCMS (ESI, m/z): 548.20 [M+H]+; ¹H NMR (300 MHz, DMSO-d₆) δ12.43 (s, 1H), 8.71 (s, 2H), 7.90 (s, 1H), 6.32-6.25 (m, 1H), 5.27 (d, J=7.3 Hz, 1H), 4.50-4.40 (m, 1H), 4.21-3.05 20 (m, 1H), 3.70-3.39 (m, 4H), 1.12 (d, J=6.4 Hz, 3H); Chiral HPLC: CHIRALPAK IG-3, 4.6*50 mm, 3 μm; detected at 254 nm.; Hex:DCM=3:1 w/0.1% DEA:EtOH=70:30, Flow rate: 1 mL/min; tR=2.058 min.
Example 13 Isomer B (13B): 5-(((S)-1-((S)-2-hydroxy-3-oxo-3-(4-(5-(trifluoromethyl)pyrimidin-2-yl)piperazin-1-yl-2,2,3,3,5,5,6,6-d₈)propoxy)propan-2-yl)amino)-4-(trifluoromethyl)pyridazin-3(2H)-one (110 mg, 19% yield, white solid); LCMS (ESI, m/z): 548.20 [M+H]⁺; ¹H NMR (300 MHZ, DMSO-d₆) δ12.44 (s, 1H), 8.71 (s, 2H), 7.89 (s, 1H), 6.32-6.24 (m, 1H), 5.27 (d, J=7.3 Hz, 1H), 4.50-4.41(m, 1H), 4.22-3.06 (m, 1H), 3.70-3.39 (m, 4H), 1.16 (d, J=6.4 Hz, 3H): Chiral HPLC: CHIRALPAK IG-3, 4.6*50 mm, 3 μm; detected at 254 nm.; Hex:DCM=3:1 w/0.1% DEA:EtOH=70:30, Flow rate: 1 mL/min; tR=2.770 min.

### Example 14: Synthesis of 5-(((2S)-1-(2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one

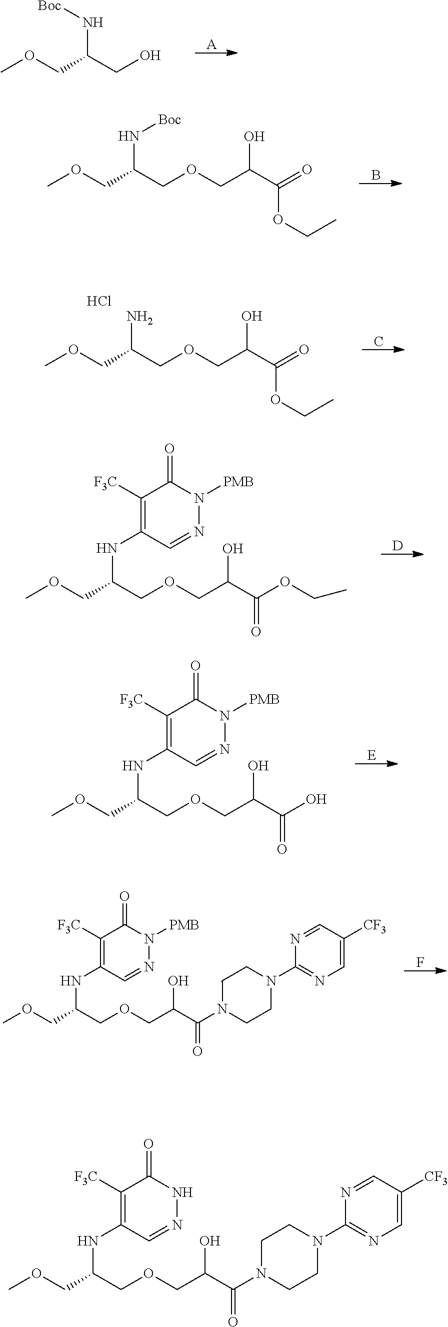

### Step A

A solution of tert-butyl (R)-(1-hydroxy-3-methoxypropan-2-yl)carbamate (2.00 g, 9.74 mmol, 1.0 equiv), methyl oxirane-2-carboxylate (3.96 g, 34.104 mmol, 3.50 equiv), and Mg(ClO₄)₂ (4.35 g, 19.488 mmol, 2.00 equiv) in EtOAc (50.00 mL) was stirred for 15 hours at 60° C. in an oil bath. 30 mL of water was added and the resulting solution was extracted with 3×50 mL of ethyl acetate. The organic layers was combined, washed with brine, dried over anhydrous sodium sulfate and concentrated under vacuum. The crude product was applied onto a silica gel column eluting with ethyl acetate/petroleum ether (3:2) to afford 380 mg (12% yield) of ethyl 3-((S)-2-((tert-butoxycarbonyl)amino)-3-methoxypropoxy)-2-hydroxypropanoate as a brown oil. LCMS (ESI, m/z): 322.20 [M+H]⁺.

### Step B

A solution of ethyl 3-((S)-2-((tert-butoxycarbonyl)amino)-3-methoxypropoxy)-2-hydroxypropanoate (380mg, 1.18 mmol, 1.0 equiv) in 4N HCl in 1,4-dioxane (4 mL) was stirred for 1 hour at room temperature. Concentration in vacuo afforded 300 mg (98% yield) of ethyl 3-((S)-2-amino-3-methoxypropoxy)-2-hydroxypropionate hydrochloride as a light brown oil. LCMS (ESI, m/z): 222.10 [M+H]⁺.

### Step C

A solution of ethyl 3-((S)-2-amino-3-methoxypropoxy)-2-hydroxypropanoate hydrochloride (300 mg, 1.16 mmol, 1.0 equiv), 5-chloro-2-(4-methoxy benzyl)-4-(trifluoromethyl)pyridazin-3(2H)-one (297 mg, 0.93 mmol, 0.80 equiv) and TEA (353 mg, 3.49 mmol, 3.0 equiv) in i-PrOH (5 mL) was stirred for 2 hours at 60° C. After concentration, the crude product was purified by silica gel chromatography with ethyl acetate/petroleum ether (7:3) to afford 100 mg (17% yield) of ethyl 2-hydroxy-3-((S)-3-methoxy-2-((1-(4-methoxybenzyl)-6-oxo-5-(trifluoromethyl)-1,6-dihydropyridazin-4-yl)amino)propoxy)propanoate as a light yellow oil. LCMS (ESI, m/z): 504.20 [M+H]⁺.

### Step D

To a solution of ethyl 2-hydroxy-3-((S)-3-methoxy-2-((1-(4-methoxy benzyl)-6-oxo-5-(trifluoromethyl)-1,6-dihydropyridazin-4-yl)amino)propoxy)propanoate (130 mg, 0.26 mmol, 1.0 equiv) in MeOH (10 mL) was added a solution of lithium hydroxide hydrate (32 mg, 0.78 mmol, 3.0 equiv) in water (2 mL). The solution was stirred for 2 hours at room temperature. The mixture was then concentrated under vacuum. The crude product was applied onto a reverse phase column eluting with MeCN:H₂O (1:1) to afford 100 mg (57% yield) of 2-hydroxy-3-((S)-3-methoxy-2-((1-(4-methoxy benzyl)-6-oxo-5-(trifluoromethyl)-1,6-dihydropyridazin-4-yl)amino)propoxy)propanoic acid as a yellow oil. LCMS (ESI, m/z): 476.16 [M+H]⁺.

### Step E

A solution of 2-hydroxy-3-((S)-3-methoxy-2-((1-(4-methoxy benzyl)-6-oxo-5-(trifluoromethyl)-1,6-dihydropyridazin-4-yl)amino)propoxy)propanoic acid (90 mg, 0.19 mmol, 1.0 equiv), 2-(piperazin-1-yl)-5-(trifluoromethyl)pyrimidine (44 mg, 0.19 mmol, 1.0 equiv), DIEA (73 mg, 0.57 mmol, 3.0 equiv) and HATU (108 mg, 0.28 mmol, 1.5 equiv) in DMF (10 mL) was stirred for 2 hours at room temperature. Water and ethyl acetate were added and the layers were separated and the aqueous layer was extracted with 3×20 ml of ethyl acetate. The organic layers were combined, dried over magnesium sulfate, filtered, and the solvent was removed in vacuo. The crude product was applied onto a reverse phase column eluting withe MeCN/Water (3:2) to afford 30 mg (23% yield) of 5-(((2S)-1-(2-hydroxy-3-oxo-3-(4-(5-(trifluoromethyl)pyrimidin-2-yl)piperazin-1-yl)propoxy)-3-methoxypropan-2-yl)amino)-2-(4-methoxy benzyl)-4-(trifluoromethyl)pyridazin-3(2H)-one as a yellow oil. LCMS (ESI, m/z): 690.15 [M+H]⁺.

### Step F

A solution of 5-(((2S)-1-(2-hydroxy-3-oxo-3-(4-(5-(trifluoromethyl)pyrimidin-2-yl)piperazin-1-yl)propoxy)-3-methoxypropan-2-yl)amino)-2-(4-methoxy benzyl)-4-(trifluoromethyl)pyridazin-3(2H)-one (20 mg, 0.029 mmol, 1.0 equiv) in TFA (3 mL) at −5° C. was treated with triflic acid (0.30 mL) and stirred for 40 min at −5° C. Water was added and the pH value of the solution was adjusted to 8 with saturated aqueous NaHCO₃. The resulting solution was extracted with 3×20 mL of EtOAc. The organic layers were combined, dried over anhydrous sodium sulfate and concentrated under vacuum. The crude product was purified by reverse phase chromatography with MeCN:Water (3:2) to afford 1.8 mg (10% yield) of 5-(((2S)-1-(2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one as a white solid. LCMS (ESI, m/z): 570.15 [M+H]⁺. ¹H NMR (300 MHz, DMSO-d₆) δ12.47 (s, 1H), 8.72 (d, J=0.9 Hz, 2H), 7.91 (d, J=3.1 Hz, 1H), 6.25-6.19 (m, 1H), 5.29 (d, J=7.3, 1.1 Hz, 1H), 4.51-4.41 (m, 1H), 4.29-4.21 (m, 1H), 3.91-3.79 (m, 4H), 3.65-3.40 (m, 10H), 3.3 (s, 3H).

Examples 15-16 in the table below were synthesized according to the procedures described for the synthesis of 5-(((2S)-1-(2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one (see Example 14) using appropriate building blocks and modified reaction conditions (such as reagent ratio, temperature, coupling conditions, and reaction time) as needed. The relative stereochemistry of the compounds was assigned by analogy to Example 1, based on the PARP7 potency of the more active diastereomer and in analogy to the Example 1A X-ray crystal structure.

| Ex. | | |
|---|---|---|
| No. | Structure | Analytical Data |
| 15 | 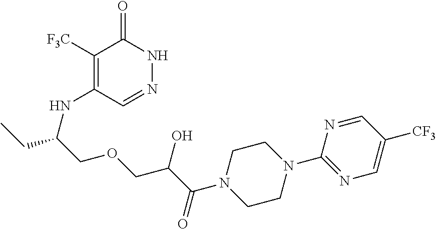 [SMILES: CC[C@@H](COCC(O)C(=O)N1CCN(c2ncc(C(F)(F)F)cn2)CC1)Nc1cn[nH]c(=O)c1C(F)(F)F] | LCMS (ESI, m/z): 554.15 [M + H]⁺; ¹HNMR (DMSO-d₆, 300 MHz) δ 12.43 (s, 1H), 8.73 (s, 2H), 7.93 (s, 1H), 6.28- 6.18 (m, 1H), 5.27 (dd, J = 7.5, 3.0 Hz, 1H), 4.51-4.42 (m, 1H), 4.05-3.98 (m, 1H), 3.95- 3.49 (m, 12H), 1.57-1.50 (m, 2H), 0.86 (t, J = 7.5 Hz, 3H). |
| | 5-(((2S)-1-(2-hydroxy-3-oxo-3-(4-(5- | |
| | (trifluoromethyl)pyrimidin-2-yl)piperazin-1- | |
| | yl)propoxy)butan-2-yl)amino)-4- | |
| | (trifluoromethyl)pyridazin-3(2H)-one | |
| 16A | 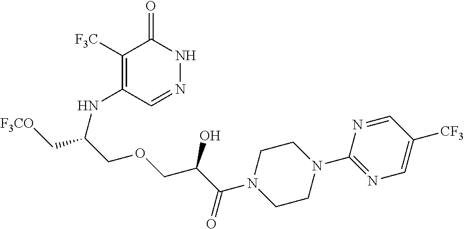 [SMILES: O=C([C@H](O)COC[C@H](COC(F)(F)F)Nc1cn[nH]c(=O)c1C(F)(F)F)N1CCN(c2ncc(C(F)(F)F)cn2)CC1] | LCMS (ESI, m/z): 624.20 [M + H ]⁺. ¹H NMR (300 MHz, DMSO-d₆) δ 12.57 (s, 1H), 8.74 (s, 2H), 7.99 (s, 1H), 6.50-6.35 (m, 1H), 5.36 (d, J = 7.1 Hz, 1H), 4.62-4.40 (m, 2H), 4.26-4.24 (m, 2H), 3.86-3.83 (m, 4H), 3.68- 3.34 (m, 8H); Chiral HPLC: CHIRALPAK IG-3, 4.6*50 mm, 3 μm; Mobile Phase A: Hex w/0.1% DEA, Mobile Phase B: EtOH; Flow rate: 1 mL/min; Gradient: 50% B for 3.4 min; 254/220 nm; rT = 1.620 min |
| | 5-(((R)-1-((R)-2-hydroxy-3-oxo-3-(4-(5- | |
| | (trifluoromethyl)pyrimidin-2-yl)piperazin-1- | |
| | yl)propoxy)-3-(trifluoromethoxy)propan-2- | |
| | yl)amino)-4-(trifluoromethyl)pyridazin-3(2H)-one | |
| 16B | 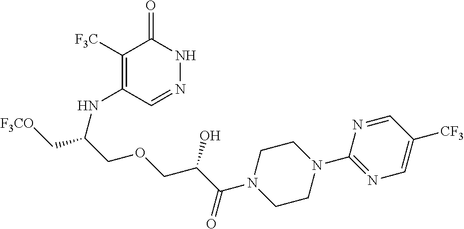 [SMILES: O=C([C@@H](O)COC[C@H](COC(F)(F)F)Nc1cn[nH]c(=O)c1C(F)(F)F)N1CCN(c2ncc(C(F)(F)F)cn2)CC1] | LCMS (ESI, m/z): 624.20 [M + H]⁺. ¹H NMR (300 MHz, DMSO-d₆) δ 12.58 (s, 1H), 8.74 (s, 2H), 7.98 (s, 1H), 6.50-6.36 (m, 1H), 5.36 (d, J = 7.2 Hz, 1H), 4.56-4.46 (m, 2H), 4.26-4.23 (m, 2H), 3.86- 3.68 (m, 4H), 3.65-3.34 (m, 8H); Chiral HPLC: CHIRALPAK IG- 3, 4.6*50 mm, 3 μm; Mobile Phase A: Hex w/0.1% DEA, Mobile Phase B: EtOH; Flow rate: 1 mL/min; Gradient: 50% B for 3.4 min; 254/220 nm; rT = 2.225 min |
| | 5-(((R)-1-((S)-2-hydroxy-3-oxo-3-(4-(5- | |
| | (trifluoromethyl)pyrimidin-2-yl)piperazin-1- | |
| | yl)propoxy)-3-(trifluoromethoxy)propan-2- | |
| | yl)amino)-4-(trifluoromethyl)pyridazin-3(2H)-one | |

### Example 17: Synthesis of 5-((S)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (17A), 5-((R)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (17B), 5-((S)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (17C), and 5-((R)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (17D)

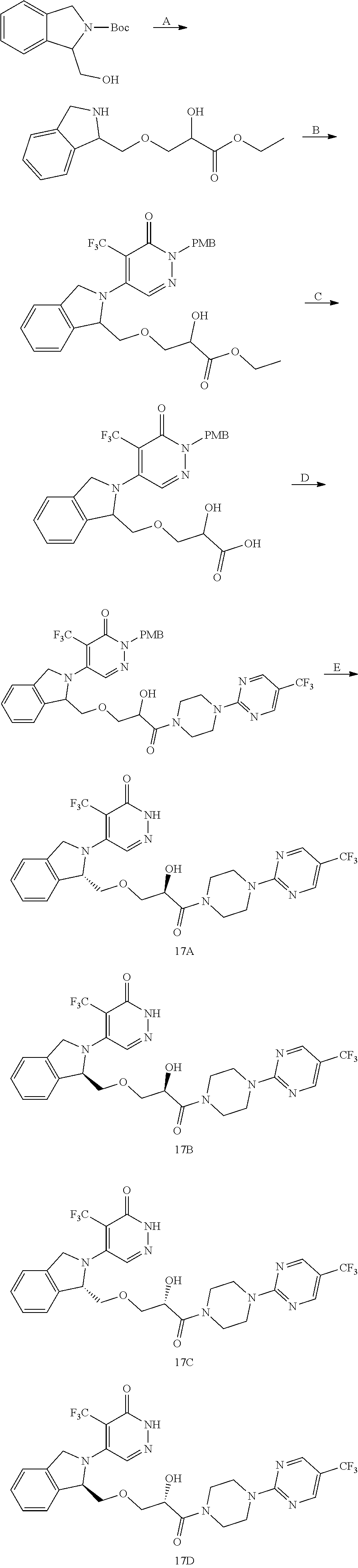

### Step A

A solution of tert-butyl 1-(hydroxymethyl)isoindoline-2-carboxylate (10.0 g), Mg(ClO₄)₂ (8.94 g, 40.1 mmol, 2.0 equiv), and ethyl oxirane-2-carboxylate (8.15 g, 70.2 mmol, 3.5 equiv) in EtOAc (50 mL) was stirred for 15 hours at 60° C. in an oil bath. 100 mL of water was added and the biphasic solution was separated and the aqueous was extracted with 3×100 mL of ethyl acetate. The organic layers were combined, dried over anhydrous sodium sulfate and concentrated under vacuum to afford 3.5 g of crude ethyl 2-hydroxy-3-(isoindolin-1-ylmethoxy)propanoate as a dark brown oil. The material was carried forward without further purification. LCMS (ESI, m/z): 266.20 [M+H]⁺.

### Step B

A solution of ethyl 2-hydroxy-3-(isoindolin-1-ylmethoxy)propanoate (3.50 g, 13.2 mmol, 1.0 equiv), 5-chloro-2-(4-methoxybenzyl)-4-(trifluoromethyl)pyridazin-3(2H)-one (4.20 g, 13.19 mmol, 1.0 equiv) and TEA (4.00 g, 39.576 mmol, 3 equiv) in i-PrOH (40 mL) was stirred for 4 hours at 60° C. 50 ml of water was added and the resulting solution was extracted with 3×50 mL of ethyl acetate. The organic layers were washed with brine, dried over anhydrous sodium sulfate and concentrated under vacuum. The crude product was purified by silica gel chromatography eluting with ethyl acetate/petroleum ether (1:1) to afford 450 mg (3% yield, 3 steps) of ethyl 2-hydroxy-3-((2-(1-(4-methoxy benzyl)-6-oxo-5-(trifluoromethyl)-1,4,5,6-tetrahydropyridazin-4-yl)isoindolin-1-yl)methoxy)propanoate as a brown solid. LCMS (ESI, m/z): 548.20[M+H]⁺.

### Step C

To a solution of ethyl 2-hydroxy-3-((2-(1-(4-methoxy benzyl)-6-oxo-5-(trifluoromethyl)-1,4,5,6-tetrahydropyridazin-4-yl)isoindolin-1-yl)methoxy)propanoate (450 mg, 0.82 mmol, 1.00 equiv) in MeOH (8 mL) at 0° C. was added a solution of lithium hydroxide hydrate (69 mg, 1.64 mmol, 2.0 equiv) in H₂O (2 mL) at 0° C. The solution was stirred for 2 hours at room temperature. The pH value of the solution was adjusted to pH 7 with aqueous IN HCl. The resulting mixture was concentrated under vacuum to afford 400 mg of crude 2-hydroxy-3-((2-(1-(4-methoxy benzyl)-6-oxo-5-(trifluoromethyl)-1,6-dihydropyridazin-4-yl)isoindolin-1-yl)methoxy)propanoic acid as a light grey solid. LCMS (ESI, m/z): 520.15 [M+H]⁺.

### Step D

A solution of 2-hydroxy-3-((2-(1-(4-methoxy benzyl)-6-oxo-5-(trifluoromethyl)-1,6-dihydropyridazin-4-yl)isoindolin-1-yl)methoxy)propanoic acid (200 mg, 0.39 mmol, 1.0 equiv), 2-(piperazin-1-yl)-5-(trifluoromethyl)pyrimidine (89 mg, 0.39 mmol, 1.0 equiv), DIEA (149 mg, 1.16 mmol, 3.0 equiv) and HATU (219 mg, 0.578 mmol, 1.5 equiv) in DMF (5 mL) was stirred for 2 hours at room temperature. 20 mL of H₂O was added and the solution was and extracted with 3×20 mL of ethyl acetate. The organic layers were washed with water and brine. The mixture was dried over anhydrous sodium sulfate and concentrated under vacuum. The crude product was purified by silica gel chromatography with ethyl acetate/petroleum ether (2:3) to afford 200 mg (35% yield) of 5-(1-((2-hydroxy-3-oxo-3-(4-(5-(trifluoromethyl)pyrimidin-2-yl)piperazin-1-yl)propoxy)methyl)isoindolin-2-yl)-2-(4-methoxybenzyl)-4-(trifluoromethyl)pyridazin-3(2H)-one as a yellow solid. LCMS (ESI, m/z): 734.25 [M+H]⁺.

### Step E

A solution of 5-(1-((2-hydroxy-3-oxo-3-(4-(5-(trifluoromethyl)pyrimidin-2-yl)piperazin-1-yl)propoxy)methyl)isoindolin-2-yl)-2-(4-methoxy benzyl)-4-(trifluoromethyl)pyridazin-3(2H)-one (320 mg, 0.44 mmol, 1.0 equiv) in TFA (8 mL) at −5° C. was treated with triflic acid (0.8 mL) and stirred for 40 min at that temperature. The resulting solution was treated with 20 mL of water. The pH value of the solution was adjusted to pH 8 with saturated aqueous Na₂CO₃ and extracted with 3×30 mL of ethyl acetate. The organic layers were combined and washed with brine. The mixture was dried over anhydrous sodium sulfate and concentrated under vacuum. The crude product was applied onto a silica gel column eluting with ethyl acetate/petroleum ether (9:1) to afford 150 mg of product. The product was further purified by chiral prep-HPLC with the following conditions: CHIRALPAK IF, 2*25 cm, 5 μm; Mobile Phase A: Hex:DCM=3:1 w/10 mM NH₃-MeOH, Mobile Phase B: EtOH; Flow rate: 20 mL/min; Gradient: 20% B for 18.5 min; 220/254 nm to afford two single isomers and a mixture of another two isomers. The mixture was futher purified by chiral prep-HPLC with the following conditions (CHIRALPAK IA, 2*25 cm, 5 μm; Mobile Phase A: MtBE w/ 10 mM NH₃-MeOH, Mobile Phase B: EtOH; Flow rate: 17 mL/min; Gradient: 20% B for 13 min; 220/254 nm to afford two separate isomers. The absolute stereochemistry of compound 17A was assigned by analogy to Example 1, based on the PARP7 potency of the more active diastereomer and in analogy to the Example 1A X-ray crystal structure. The stereochemistry of 17B, 17C, and 17D was arbitrarily assigned.
Example 17 Isomer A (17A): 5-((S)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (16 mg, 6% yield) as a white solid. LCMS (ESI, m/z): 614.20 [M+H]⁺; ¹H-NMR (300 MHZ, DMSO-d₆) δ12.49 (s, 1H), 8.73 (s, 2H), 8.18 (s, 1H), 7.39-7.23 (m, 4H), 5.87 (s, 1H), 5.20-5.05 (m, 2H), 4.49-4.39 (m, 2H), 3.80-3.48 (m, 12H). Chiral HPLC: CHIRALPAK IF-3, 4.6*50 mm 3 μ; Hex:DCM=3:1 w/0.1% DEA:EtOH=4:1; Flow rate 1 mL/min), rT=2.040 min.
Example 17 Isomer B (17B—stereochemistry arbitrarily assigned): 5-((R)-1 -(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (16 mg, 6% yield) as a white solid. LCMS (ESI, m/z): 614.20 [M+H]⁺; ¹HNMR (300 MHZ, DMSO-d₆) δ12.49 (s, 1H), 8.73 (s, 2H), 8.15 (s, 1H), 7.42-7.25 (m, 4H), 5.87 (s, 1H), 5.25-5.01 (m, 2H), 4.48-4.38 (m, , 2H), 3.90-3.40 (m, 12H). Chiral HPLC: CHIRALPAK IA-3, 4.6*50 mm, 3 μm; MtBE w/0.1% DEA:EtOH=80:20; Flow rate 1 mL/min; rT=1.454 min.
Example 17 Isomer C (17C—stereochemistry arbitrarily assigned): 5-((S)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (13mg, 5% yield) as a white solid. LCMS (ESI, m/z): 614.20 [M+H]⁺; ¹H NMR (300 MHZ, DMSO-d₆) 67 12.49 (s, 1H), 8.73 (s, 2H), 8.15 (s, 1H), 7.50-7.21 (m, 4H), 5.86 (s, 1H), 5.19 (d, J=7.3 Hz, 1H), 5.03 (d, J=14.2 Hz, 1H), 4.48-4.32 (m, 2H), 3.80-3.31 (m, 12H). Chiral HPLC: CHIRALPAK IA-3, 4.6*50 mm, 3 μm; MtBE w/0.1% DEA:EtOH=80:20; Flow rate 1 mL/min; rT=1.789 min.
Example 17 Isomer D (17D—stereochemistry arbitrarily assigned): 5-((R)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one (13 mg, 5% yield) as a white solid. LCMS (ESI, m/z): 614.20 [M+H]⁺; ¹H-NMR(300 MHZ, DMSO-d₆) δ12.49 (s, 1H), 8.73 (s, 2H), 8.15 (s, 1H), 7.42-7.20 (m, 4H), 5.87 (s, 1H), 5.18 (d, J=7.2 Hz, 1H), 5.03 (d, J=15.2 Hz, 1H), 4.48-4.38 (m, 2H), 3.90-3.35 (m, 12H). Chiral HPLC: CHIRALPAK IF-3, 4.6*50 mm 3 μm; Hex:DCM=3:1 w/0.1% DEA:EtOH=4:1; Flow rate 1 mL/min), rT=3.288 min.

### Example 18: Synthesis of 5-((2S)-2-((2-hydroxy-3-oxo-3-(4-(5-(trifluoromethyl)pyrimidin-2-yl)piperazin-1-yl)propoxy)methyl)azetidin-1-yl)-4-(trifluoromethyl)pyridazin-3(2H)-one

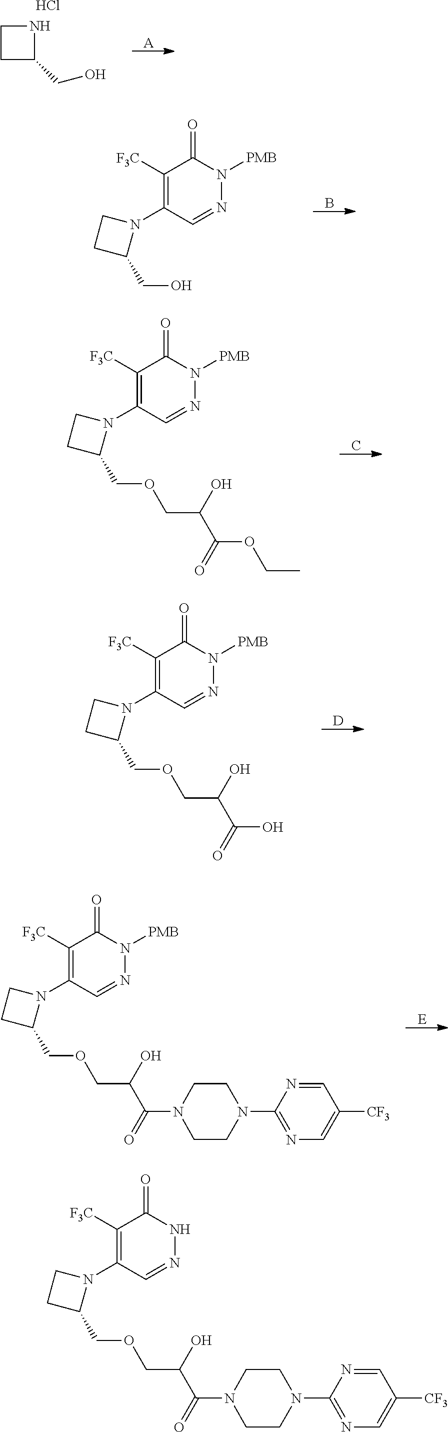

### Step A

A solution of (S)-azetidin-2-ylmethanol hydrochloride (1.20 g, 9.71 mmol, 1.0 equiv), TEA (2.80 mL), and 5-chloro-2-(4-methoxy benzyl)-4-(trifluoromethyl)pyridazin-3(2H)-one (3.20 g, 10.0 mmol, 1.03 equiv) in ethanol (20 mL) was stirred for 1 hour at 60° C. After cooling to room temperature, the solution was concentrated under vacuum. The crude product was purified by reverse phase chromatography eluting with H₂O/CH₃CN to afford 2 g (55% yield) of (S)-5-(2-(hydroxymethyl)azetidin-1-yl)-2-(4-methoxy benzyl)-4-(trifluoromethyl)pyridazin-3(2H)-one as a white solid. LCMS (ESI, m/z): 370.15 [M+H]⁺.

### Step B

A solution of ethyl oxirane-2-carboxylate (980 mg, 8.44 mmol, 2.0 equiv), BF₃ Et₂O (300 mg, 2.11 mmol, 0.5 equiv), and (S)-5-(2-(hydroxymethyl)azetidin-1-yl)-2-(4-methoxy benzyl)-4-(trifluoromethyl)pyridazin-3(2H)-one (1.56 g, 4.22 mmol, 1.00 equiv) in dichloromethane (100 mL) was stirred for 15 hours at room temperature. Water (50 mL) was added and the layers were separated. The aqueous layer was extracted with 3×50 mL dichloromethane. The organic layers were combined, dried over anhydrous sodium sulfate and concentrated under vacuum. The crude product was purified by reverse phase chromatography eluting with H₂O/CH₃CN to afford 100 mg (5% yield) of ethyl 2-hydroxy-3-(((S)-1-(1-(4-methoxy benzyl)-6-oxo-5-(trifluoromethyl)-1,6-dihydropyridazin-4-yl)azetidin-2-yl)methoxy)propanoate as a yellow oil. LCMS (ESI, m/z): 486.15 [M+H]⁺.

### Step C

To the solution of ethyl 2-hydroxy-3-(((S)-1-(1-(4-methoxy benzyl)-6-oxo-5-(trifluoromethyl)-1,6-dihydropyridazin-4-yl)azetidin-2-yl)methoxy)propanoate (90 mg, 0.18 mmol, 1.00 equiv) in methanol (1 mL) was added a solution of lithium hydroxide hydrate (12 mg, 0.29 mmol, 1.54 equiv) in H₂O (1 mL). The resulting solution was stirred for 1 hour at room temperature. The mixture was concentrated under vacuum and the crude product was purified by reverse phase chromatography eluting with H₂O/CH₃CN to afford 50 mg (59% yield) of 2-hydroxy-3-(((S)-1-(1-(4-methoxy benzyl)-6-oxo-5-(trifluoromethyl)-1,6-dihydropyridazin-4-yl)azetidin-2-yl)methoxy)propanoic acid as a yellow oil. LCMS (ESI, m/z): 458.10 [M+H]⁺.

### Step D

A solution of 2-hydroxy-3-(((S)-1-(1-(4-methoxy benzyl)-6-oxo-5-(trifluoromethyl)-1,6-dihydropyridazin-4-yl)azetidin-2-yl)methoxy)propanoic acid (40 mg, 0.087 mmol, 1.0 equiv), HATU (32 mg, 0.084 mmol, 0.96 equiv), DIEA (20 mg, 0.16 mmol, 1.8 equiv), and 2-(piperazin-1-yl)-5-(trifluoromethyl)pyrimidine (20 mg, 0.086 mmol, 0.98 equiv) in DMF (2 mL) was stirred for 1 hour at room temperature. The crude product was purified directly by reverse phase chromatography eluting with H₂O/CH₃CN to afford 22 mg (37% yield) of 5-((2S)-2-((2-hydroxy-3-oxo-3-(4-(5-(trifluoromethyl)pyrimidin-2-yl)piperazin-1-yl)propoxy)methyl)azetidin-1-yl)-2-(4-methoxy benzyl)-4-(trifluoromethyl)pyridazin-3(2H)-one as a yellow oil. LCMS (ESI, m/z): 672.20 [M+H]⁺.

### Step E

A solution of 5-((2S)-2-((2-hydroxy-3-oxo-3-(4-(5-(trifluoromethyl)pyrimidin-2-yl)piperazin-1-yl)propoxy)methyl)azetidin-1-yl)-2-(4-methoxy benzyl)-4-(trifluoromethyl)pyridazin-3(2H)-one (17 mg, 0.025 mmol, 1.0 equiv) in trifluoromethanesulfonic acid (0.1 mL) and trifluoroacetic acid (1 mL) was stirred for 1 h at 0° C. Water (5 mL) was added and the pH value of the solution was adjusted to 7-8 with saturated K₂CO₃ aqueous solution. The resulting solution was extracted with 3×20 mL of ethyl acetate. The organic layers were combined, dried over anhydrous sodium sulfate and concentrated under vacuum. The crude product was purified by reverse phase chromatography eluting with H₂O/CH₃CN to afford 2.6 mg (18% yield) of 5-((2S)-2-((2-hydroxy-3-oxo-3-(4-(5-(trifluoromethyl)pyrimidin-2-yl)piperazin-1-yl)propoxy)methyl)azetidin-1-yl)-4-(trifluoromethyl)pyridazin-3(2H)-one as a white solid. LCMS (ESI, m/z): 552.15 [M+H]⁺. ¹H NMR (DMSO-d₆, 300 MHz) δ12.33 (br s, 1H), 8.73 (s, 2H), 7.66 (s, 1H), 5.32 (br, 1H), 4.84-4.79 (m, 1H), 4.55-4.51 (m, 1H), 4.35-4.29 (m, 1H), 3.87-3.50 (m, 12H), 3.48-3.40 (m, 1H), 2.50-2.49 (m, 1H), 1.99-1.96 (m, 1H).

### Example A. Enzymatic Assay for Inhibition of PARP7

Displacement of Probe A, a biotinylated probe binding to the TIPARP active site, was measured using a time-resolved fluorescence energy transfer (TR-FRET) assay. 20 nL of a dose response curve of each test compound was spotted in black 384-well polystyrene proxiplates (Perkin Elmer) using a Mosquito (TTP Labtech). Reactions were performed in a 8 μL volume by adding 6 μL of TIPARP and Probe A in assay buffer (20 mM HEPES pH=8, 100 mM NaCl, 0.1% bovine serum albumin, 2 mM DTT and 0.002% Tween20), incubating with test compound at 25° C. for 30 min, then adding 2 μL of ULight-anti 6xHis and LANCE Eu-W1024 labeled streptavidin (Perkin Elmer). The final concentrations of

TIPARP and Probe A were 6 nM and 2 nM, respectively. The final concentration of ULight-anti 6xHis and LANCE Eu-W1024 labeled streptavidin were 4 nM and 0.25 nM, respectively Reactions were incubated at 25° C. for an additional 30 min, then read on an Envision platereader equipped with a LANCE/DELFIA top mirror (Perkin Elmer) using excitation of 320 nm and emission of 615 nm and 665 nM with a 90 μs delay. The ratio of the 665/615 nm emission were calculated for each well to determine the amount of complex of TIPARP and Probe A in each well. Control wells containing a negative control of 0.25% DMSO vehicle or a positive control of 100 M 5-(5-(piperidin-4-yloxy)isoindolin-2-yl)-4-(trifluoromethyl)pyridazin-3(2H)-one were used to calculate the % inhibition as described below:

$$\% inhibition = 100 \times \frac{{TRF}_{cmpd} - {TRF}_{min}}{{TRF}_{max} - {TRF}_{min}}$$

where TRF_{cmpd} is the TR-FRET ratio from the compound treated well. TRFₘᵢₙ is the TR-FRET ratio from the 5-(5-(piperidin-4-yloxy)isoindolin-2-yl)-4-(trifluoromethyl)pyridazin-3(2H)-one -treated positive control well and TREₘₐₓ is the TR-FRET ratio from the DMSO-treated negative control well.

The % inhibition values were plotted as a function of compound concentration and the following 4-parameter fit was applied to derive the IC₅₀ values:

$$Y = Bottom + \frac{( Top - Bottom )}{( 1 + {( \frac{X}{{IC}_{50}} )}^{Hill Coefficient}}$$

where top and bottom are normally allowed to float, but may be fixed at 100 or 0 respectively in a 3-parameter fit. The Hill Coefficient is normally allowed to float but may also be fixed at 1 in a 3-parameter fit. Y is the % inhibition and X is the compound concentration.

### Synthesis of Probe A

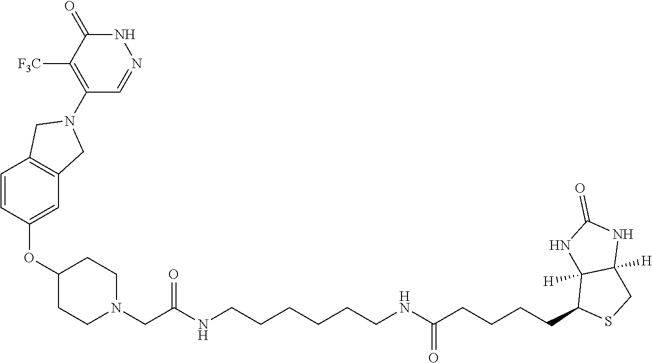 [SMILES: O=C(CCCC[C@@H]1SC[C@@H]2NC(=O)N[C@@H]21)NCCCCCCNC(=O)CN1CCC(Oc2ccc3c(c2)CN(c2cn[nH]c(=O)c2C(F)(F)F)C3)CC1]

### Step A

A solution of 5-chloro-4-(trifluoromethyl)-2-[[2-(trimethylsilyl)ethoxy]methyl]-2,3-dihydropyridazin-3-one (2.8 g, 8.52 mmol, 1.00 equiv), 2,3-dihydro-1H-isoindol-5-ol hydrobromide (4.27 g, 19.76 mmol, 1.00 equiv), and TEA (10 mL) in ethanol (40 mL) was stirred for 1 h at 60° C. The resulting solution was extracted with 2×100 mL of ethyl acetate and the organic layers combined and concentrated under reduced pressure to afford 4.5 g of 5-(5-hydroxy-2,3-dihydro-1H-isoindol-2-yl)-4-(trifluoromethyl)-2-[[2-(trimethylsilyl)ethoxy]methyl]-2,3-dihydropyridazin-3-one as a yellow oil. LCMS: [M+H]⁺428.23.

### Step B

A solution of 5-(5-hydroxy-2,3-dihydro-1H-isoindol-2-yl)-4-(trifluoromethyl)-2-[[2-(trimethylsilyl)ethoxy]methyl]-2,3-dihydropyridazin-3-one (4.5 g, 10.53 mmol, 1.00 equiv), tert-butyl 4-iodopiperidine-1-carboxylate (20 g, 64.28 mmol, 8.00 equiv), potassium carbonate (15 g, 108.53 mmol, 10.00 equiv), and DMF (50 mL) was stirred for 2 days at 80° C. The resulting solution was extracted with 2×200 mL of ethyl acetate and the organic layers combined and concentrated under reduced pressure. The residue was applied onto a silica gel column eluting with ethyl acetate/petroleum ether to afford tert-butyl 4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidine-1-carboxylate (2 g, 31%) as a yellow oil. LCMS: [M+H]⁺611.15.

### Step C

A solution of tert-butyl 4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidine-1-carboxylate (2 g, 3.27 mmol, 1.00 equiv), dioxane/HCl (5 mL), and dioxane (45 mL) was stirred for 6 h at 25° C. The resulting mixture was concentrated under reduced pressure. The residue was applied onto a silica gel column and eluted with ethyl acetate/petroleum ether to afford 1 g of 5-[5-(piperidin-4-yloxy)-2,3-dihydro-1H-isoindol-2-yl]-4-(trifluoromethyl)-2-[[2-(trimethylsilyl)ethoxy]methyl]-2,3-dihydropyridazin-3-one as a yellow oil. LCMS: [M+H]⁺511.28.

### Step D

A solution of 5-[5-(piperidin-4-yloxy)-2,3-dihydro-1H-isoindol-2-yl]-4-(trifluoromethyl)-2-[[2-(trimethylsilyl)ethoxy]methyl]-2,3-dihydropyridazin-3-one (1 g, 1.96 mmol, 1.00 equiv), tert-butyl 2-chloroacetate (450 mg, 2.99 mmol, 3.00 equiv), DIPEA (5 mL), and dichloromethane (10 mL) was stirred overnight at 25° C. The residue was purified by C18 reverse phase chromatography eluting with H₂O/CH₃CN to afford tert-butyl 2-[4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidin-1-yl]acetate (540 mg, 44%) as a yellow oil. LCMS: [M+H]⁺625.20.

### Step E

A solution of tert-butyl 2-[4-([2-[6-oxo-5-(trifluoromethyl)-1-[[2-(trimethylsilyl)ethoxy]methyl]-1,6-dihydropyridazin-4-yl]-2,3-dihydro-1H-isoindol-5-yl]oxy)piperidin-1-yl]acetate (540 mg, 0.86 mmol, 1.00 equiv) and dioxane/HCl (8 mL) was stirred overnight at 25° C. The resulting mixture was concentrated under reduced pressure. The residue was purified by C₁₈ reverse phase chromatography eluting with H₂O/CH₃CN to afford 200 mg (53%) of 2-[4-([2-[6-oxo-5-(trifluoromethyl)-1,6-dihydropyridazin-4-yl]-2,3-dihydro-1H-isoindol-5-yl]oxy)piperidin-1-yl]hydrochloride as a white solid. LCMS: [M+H]⁺439.31.

### Step F

A solution of 5-[(3aS,4S,6aR)-2-oxo-hexahydro-1H-thieno[3,4-d]imidazolidin-4-yl]pentanoic acid (reagent was purchased from Beijing Dragon Rui Trading Company, 976 mg, 3.99 mmol, 1.00 equiv), DIPEA (1.55 g, 11.99 mmol, 3.00 equiv), HATU (1.82 g, 4.79 mmol, 1.20 equiv), tert-butyl N-(6-aminohexyl)carbamate (864 mg, 3.99 mmol, 1.00 equiv) in DMF (15 mL) was stirred overnight at 25° C. The reaction was then quenched by the addition of 50 ml of water. The solids were collected by filtration to afford 1.5 g (85%) of tert-butyl N-(6-[5-[(3aS,4S,6aR)-2-oxo-hexahydro-1H-thieno[3,4-d]imidazolidin-4-yl]pentanamido]hexyl)carbamate as a white solid. LCMS: [M+H]⁺443.26.

### Step G

A solution of tert-butyl N-(6-[5-[(3aS,4S,6aR)-2-oxo-hexahydro-1H-thieno[3,4-d]imidazolidin-4-yl]pentanamido]hexyl)carbamate (800 mg, 1.81 mmol, 1.00 equiv) in hydrogen chloride/dioxane (20 mL) was stirred overnight at 25° C. The resulting mixture was concentrated under reduced pressure to afford 600 mg (88%) of 5-[(3aS,4S,6aR)-2-Oxo-hexahydro-1H-thieno[3,4-d]imidazolidin-4-yl]-N-(6-aminohexyl)pentanamide hydrochloride as a gray crude oil. LCMS: [M+H]⁺343.21.

### Step H

A solution of 2-[4-([2-[6-oxo-5-(trifluoromethyl)-1,6-dihydropyridazin-4-yl]-2,3-dihydro-1H-isoindol-5-yl]oxy)piperidin-1-yl]hydrochloride (175 mg, 0.40 mmol, 1.00 equiv), DIPEA (258 mg, 2.00 mmol, 5.00 equiv), HATU (228 mg, 0.60 mmol, 1.50 equiv), 5-[(3aS,4S,6aR)-2-oxo-hexahydro-1H-thieno[3,4-d]imidazolidin-4-yl]-N-(6-aminohexyl)pentanamide hydrochloride (228 mg, 0.60 mmol, 1.50 equiv) in DMF (3 mL) was stirred for 4 h at 25° C. The crude product was purified by C18 reverse phase chromatography eluting with H₂O/CH₃CN to afford 5-[(3aS,4S,6aR)-2-oxo-hexahydro-1H-thieno[3,4-d]imidazolidin-4-yl]-N-(6-[2-[4-([2-[6-oxo-5-(trifluoromethyl)-1,6-dihydropyridazin-4-yl]-2,3-dihydro-1H-isoindol-5-yl]oxy)piperidin-1-yl]acetamido]hexyl)pentanamide as a white solid (118.3 mg, 39%). LCMS: [M+H]⁺763.35.
¹H NMR (DMSO-d₆, 400 MHZ) δ: 12.52 (s, 1H), 7.98 (s, 1H), 7.81-7.68 (m, 2H), 7.26 (d, J=8.4 Hz, 1H), 7.00 (d, J=2.2 Hz, 1H), 6.91 (dd, J=8.4, 2.3 Hz, 1H), 6.45-6.39 (m, 1H), 6.36 (s, 1H), 4.91 (d, J=6.1 Hz, 4H), 4.45 (m, 1H), 4.26 (m, 1H), 4.17-4.08 (m, 1H), 3.14-2.96 (m, 5H), 2.91 (s, 2H), 2.82 (dd, J=12.4, 5.1 Hz, 1H), 2.73-2.63 (m, 2H), 2.58 (d, J=12.4 Hz, 1H), 2.33 (ddd, J=11.8, 9.4, 3.1 Hz, 2H), 2.11-1.90 (m, 4H), 1.76-1.54 (m, 3H), 1.57-1.20 (m, 13H).

IC₅₀ data for the Example compounds is provided below in Table A-1 (“+” is <0.1 μM).

TABLE A-1
IC₅₀ Data for Example Compounds

| Example | |
|---|---|
| No. | IC₅₀ |
| 1A | + |
| 1B | + |
| 2A | + |
| 2B | + |
| 3A | + |
| 3B | + |
| 4A | + |
| 4B | + |
| 5 | + |
| 6A | + |
| 6B | + |
| 7A | + |
| 7B | + |
| 8A | + |
| 8B | + |
| 9A | + |
| 10A | + |
| 11A | + |
| 12A | + |
| 12B | + |
| 13A | + |
| 13B | + |
| 14 | + |
| 15 | + |
| 16A | + |
| 16B | + |
| 17A | + |
| 17B | + |
| 17C | + |
| 17D | + |
| 18 | + |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound having Formula I: 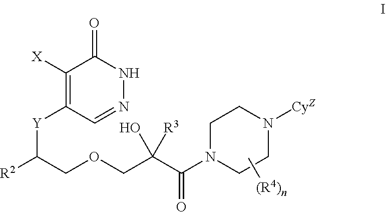
or a pharmaceutically acceptable salt thereof, wherein:
X is halo or CF₃;
Y is NR¹ or 0;
R¹ is H or C₁₋₆ alkyl;
R² is H or C₁₋₆ alkyl; wherein said C₁₋₆ alkyl is optionally substituted with OR^{a};
or R¹ and R² together with the atoms to which they are attached form a 4-10 membered heterocycloalkyl group; wherein the 4-10 membered heterocycloalkyl group has at least one ring-forming carbon atom and 1, 2, or 3 ring-forming heteroatoms independently selected from N, O, and S; wherein a ring-forming carbon atom of the 4-10 membered heterocycloalkyl group is optionally substituted by oxo to form a carbonyl group; and wherein the 4-10 membered heterocycloalkyl group is optionally substituted with 1, 2, 3, or 4 substituents each independently selected from halo, D, OH, C₁₋₆ alkyl, C₁₋₆ haloalkyl, C₁₋₆ alkoxy, C₁₋₆ alkylamino, and
C₁₋₆ dialkylamino;
R³ is H or C₁₋₆ alkyl;
each R⁴ is independently selected from D, C₁₋₆ alkyl, C₁₋₆ haloalkyl, and C₁₋₆ alkoxy;
Cy^{Z} is a 5-10 membered heteroaryl group substituted with R^{z};
R^{Z} is halo, CN, C₁₋₆ alkyl, or C₁₋₃ haloalkyl;
R^{a} is selected from H, C₁₋₆ alkyl, and C₁₋₆ haloalkyl; and
n is an integer from 0 to 8.

2. The compound of claim 1, or a pharmaceutically acceptable salt thereof, wherein X is CF₃.

3. The compound of claim 1, or a pharmaceutically acceptable salt thereof, wherein X is halo.

4. The compound of claim 1, or a pharmaceutically acceptable salt thereof, wherein X is Br or CF₃.

5. The compound of any one of claims 1-4, or a pharmaceutically acceptable salt thereof, wherein Y is NR¹.

6. The compound of any one of claims 1-4, or a pharmaceutically acceptable salt thereof, wherein Y is O.

7. The compound of any one of claims 1-6, or a pharmaceutically acceptable salt thereof, wherein R¹ is H.

8. The compound of any one of claims 1-7, or a pharmaceutically acceptable salt thereof, wherein R² is C₁₋₆ alkyl, wherein said C₁₋₆ alkyl is optionally substituted with OR^{a}.

9. The compound of any one of claims 1-7, or a pharmaceutically acceptable salt thereof, wherein R² is H or C₁₋₃ alkyl, wherein said C₁₋₃ alkyl is optionally substituted with OR^{a}.

10. The compound of any one of claims 1-7, or a pharmaceutically acceptable salt thereof, wherein R² is H, methyl, or ethyl, wherein said methyl and ethyl are each optionally substituted with methoxy or trifluoromethoxy.

11. The compound of any one of claims 1-7, or a pharmaceutically acceptable salt thereof, wherein R² is H, methyl, ethyl, methoxymethyl, or trifluoromethoxymethyl.

12. The compound of any one of claims 1-7, or a pharmaceutically acceptable salt thereof, wherein R² is methyl.

13. The compound of any one of claims 1-6, or a pharmaceutically acceptable salt thereof, wherein R¹ and R² together with the atoms to which they are attached form a 4-10 membered heterocycloalkyl group; wherein the 4-10 membered heterocycloalkyl group is optionally substituted with 1 or 2 substituents each independently selected from halo, D, OH, C₁₋₆ alkyl, C₁₋₆ haloalkyl, C₁₋₆ alkoxy, and C₁₋₆ alkylamino.

14. The compound of any one of claims 1-6, or a pharmaceutically acceptable salt thereof, wherein R¹ and R² together with the atoms to which they are attached form an azetidine ring or isoindoline ring; each of which is optionally substituted with 1 or 2 substituents each independently selected from halo, D, OH, C₁₋₆ alkyl, C₁₋₆ haloalkyl, C₁₋₆ alkoxy, and C₁₋₆ alkylamino.

15. The compound of any one of claims 1-6, or a pharmaceutically acceptable salt thereof, wherein R¹ and R² together with the atoms to which they are attached form an azetidine ring or isoindoline ring.

16. The compound of any one of claims 1-15, or a pharmaceutically acceptable salt thereof, wherein R³ is H or C₁₋₆ alkyl.

17. The compound of any one of claims 1-15, or a pharmaceutically acceptable salt thereof, wherein R³ is H or methyl.

18. The compound of any one of claims 1-15, or a pharmaceutically acceptable salt thereof, wherein R³ is H.

19. The compound of any one of claims 1-18, or a pharmaceutically acceptable salt thereof, wherein each R⁴ is D.

20. The compound of any one of claims 1-19, or a pharmaceutically acceptable salt thereof, wherein n is 0.

21. The compound of any one of claims 1-19, or a pharmaceutically acceptable salt thereof, wherein n is 8.

22. The compound of any one of claims 1-18, or a pharmaceutically acceptable salt thereof, wherein n is 8; and each R⁴ is D.

23. The compound of any one of claims 1-22, or a pharmaceutically acceptable salt thereof, wherein Cy^{Z} is a 5-6 membered heteroaryl group substituted with R^{Z}.

24. The compound of any one of claims 1-22, or a pharmaceutically acceptable salt thereof, wherein Cy^{Z} is a pyrimidinyl, pyrazinyl, pyridinyl, or thiazolyl group substituted with R^{Z}.

25. The compound of any one of claims 1-22, or a pharmaceutically acceptable salt thereof, wherein Cy^{Z} is selected from 5-(trifluoromethyl)pyrimidin-2-yl, 5-(trifluoromethyl)thiazol-2-yl, 5-(trifluoromethyl)pyrazin-2-yl, 5-cyanopyridin-2-yl, 5-(difluoromethyl)pyrimidin-2-yl, and 5-chloropyrimidin-2-yl.

26. The compound of any one of claims 1-25, or a pharmaceutically acceptable salt thereof, wherein R^{Z} is halo, CN or C₁₋₃ haloalkyl.

27. The compound of any one of claims 1-25, or a pharmaceutically acceptable salt thereof, wherein R^{Z} is C₁₋₃ haloalkyl.

28. The compound of any one of claims 1-25, or a pharmaceutically acceptable salt thereof, wherein R^{Z} is selected from CF₃, CN, CF₂H, and Cl.

29. The compound of any one of claims 1-28, having Formula II: 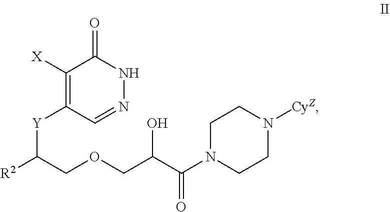
or a pharmaceutically acceptable salt thereof.

30. The compound of any one of claims 1-28, having Formula IIIa: 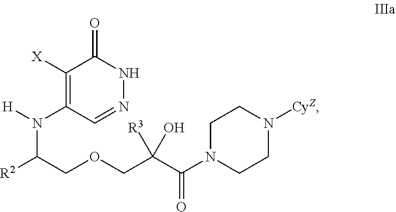
or a pharmaceutically acceptable salt thereof.

31. The compound of any one of claims 1-28, having Formula IIIb: 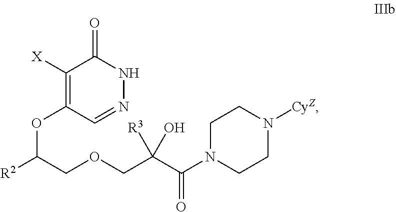
or a pharmaceutically acceptable salt thereof.

32. The compound of any one of claims 1-28, having Formula IV: 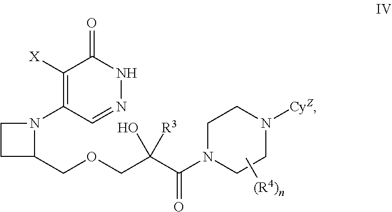
or a pharmaceutically acceptable salt thereof.

33. The compound of any one of claims 1-28, having Formula V: 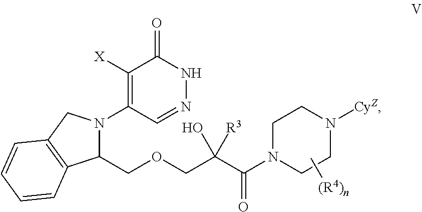
or a pharmaceutically acceptable salt thereof.

34. The compound of any one of claims 1-28, having Formula VI: 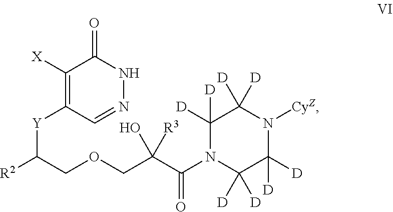
or a pharmaceutically acceptable salt thereof.

35. The compound of claim 1, or a pharmaceutically acceptable salt thereof, wherein:
X is halo or CF₃;
Y is NR¹ or O;
R¹ is H;
R² is H or C₁₋₆ alkyl, wherein said C₁₋₆ alkyl is optionally substituted with OR^{a};
or R¹ and R² together with the atoms to which they are attached form a 4-10 membered heterocycloalkyl group; wherein the 4-10 membered heterocycloalkyl group has at least one ring-forming carbon atom and 1, 2, or 3 ring-forming heteroatoms independently selected from N, O, and S;
R³ is H or C₁₋₆ alkyl;
each R⁴ is D;
Cy^{Z} is a 5-10 membered heteroaryl group substituted with R^{Z};
R^{Z} is halo, CN, or C₁₋₃ haloalkyl;
R^{a} is selected from C₁₋₆ alkyl and C₁₋₆ haloalkyl; and
n is 0 or 8.

36. The compound of claim 1, selected from:
5-(((S)-1-((R)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one;
5-(((S)-1-((S)-2-hydroxy-3-oxo-3-(4-(5-(trifluoromethyl)pyrimidin-2-yl)piperazin-1-yl)propoxy)propan-2-yl)amino)-4-(trifluoromethyl)pyridazin-3(2H)-one;
5-(((S)-1-((R)-2-hydroxy-3-oxo-3-(4-(5-(trifluoromethyl)pyrazin-2-yl)piperazin-1-yl)propoxy)propan-2-yl)amino)-4-(trifluoromethyl)pyridazin-3(2H)-one;
5-(((S)-1-((S)-2-hydroxy-3-oxo-3-(4-(5-(trifluoromethyl)pyrazin-2-yl)piperazin-1-yl)propoxy)propan-2-yl)amino)-4-(trifluoromethyl)pyridazin-3(2H)-one;
5-((S)-1-((R)-2-hydroxy-3-oxo-3-(4-(5-(trifluoromethyl)thiazol-2-yl)piperazin-1-yl)propoxy)propan-2-ylamino)-4-(trifluoromethyl)pyridazin-3(2H)-one;
5-((S)-1-((S)-2-hydroxy-3-oxo-3-(4-(5-(trifluoromethyl)thiazol-2-yl)piperazin-1-yl)propoxy)propan-2-ylamino)-4-(trifluoromethyl)pyridazin-3(2H)-one;
5-(((S)-1-((R)-3-(4-(5-(difluoromethyl)pyrimidin-2-yl)piperazin-1-yl)-2-hydroxy-3-oxopropoxy)propan-2-yl)amino)-4-(trifluoromethyl)pyridazin-3(2H)-one;
5-(((S)-1-((S)-3-(4-(5-(difluoromethyl)pyrimidin-2-yl)piperazin-1-yl)-2-hydroxy-3-oxopropoxy)propan-2-yl)amino)-4-(trifluoromethyl)pyridazin-3(2H)-one;
5-(((2S)-1-(2-hydroxy-2-methyl-3-oxo-3-(4-(5-(trifluoromethyl)pyrimidin-2-yl)piperazin-1-yl)propoxy)propan-2-yl)amino)-4-(trifluoromethyl)pyridazin-3(2H)-one;
5-(((S)-1-((S)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one;
5-(((S)-1-((R)-3-(4-(5-chloropyrimidin-2-yl)piperazin-1-yl)-2-hydroxy-3-oxopropoxy)propan-2-yl)amino)-4-(trifluoromethyl)pyridazin-3(2H)-one;
5-(((S)-1-((S)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one;
5-(((S)-1-((R)-2-hydroxy-3-oxo-3-(4-(5-(trifluoromethyl)pyrimidin-2-yl)piperazin-1-yl)propoxy)propan-2-yl)oxy)-4-(trifluoromethyl)pyridazin-3(2H)-one;
5-(((S)-1-((R)-2-hydroxy-3-oxo-3-(4-(5-(trifluoromethyl)pyrazin-2-yl)piperazin-1-yl)propoxy)propan-2-yl)oxy)-4-(trifluoromethyl)pyridazin-3(2H)-one;
5-(((S)-1-((S)-2-hydroxy-3-oxo-3-(4-(5-(trifluoromethyl)pyrazin-2-yl)piperazin-1-yl)propoxy)propan-2-yl)oxy)-4-(trifluoromethyl)pyridazin-3(2H)-one;
5-(((S)-1-((R)-3-(4-(5-chloropyrimidin-2-yl)piperazin-1-yl)-2-hydroxy-3-oxopropoxy)propan-2-yl)oxy)-4-(trifluoromethyl)pyridazin-3(2H)-one;
6-(4-((R)-2-hydroxy-3-((S)-2-((6-oxo-5-(trifluoromethyl)-1,6-dihydropyridazin-4-yl)oxy)propoxy)propanoyl)piperazin-1-yl)nicotinonitrile;
5-(((S)-1-((R)-3-(4-(5-(difluoromethyl)pyrimidin-2-yl)piperazin-1-yl)-2-hydroxy-3-oxopropoxy)propan-2-yl)oxy)-4-(trifluoromethyl)pyridazin-3(2H)-one;
4-bromo-5-(((S)-1-((S)-2-hydroxy-3-oxo-3-(4-(5-(trifluoromethyl)pyrimidin-2-yl)piperazin-1-yl)propoxy)propan-2-yl)oxy)pyridazin-3(2H)-one;
4-bromo-5-(((S)-1-((R)-2-hydroxy-3-oxo-3-(4-(5-(trifluoromethyl)pyrimidin-2-yl)piperazin-1-yl)propoxy)propan-2-yl)oxy)pyridazin-3(2H)-one;
5-(((S)-1-((S)-2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-4-(trifluoromethyl)pyridazin-3(2H)-one;
5-(((S)-1-((R)-2-hydroxy-3-oxo-3-(4-(5-(trifluoromethyl)pyrimidin-2-yl)piperazin-1-yl-2,2,3,3,5,5,6,6-d8)propoxy)propan-2-yl)amino)-4-(trifluoromethyl)pyridazin-3(2H)-one;
5-(((2S)-1-(2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one;
5-(((2S)-1-(2-hydroxy-3-oxo-3-(4-(5-(trifluoromethyl)pyrimidin-2-yl)piperazin-1-yl)propoxy)butan-2-yl)amino)-4-(trifluoromethyl)pyridazin-3(2H)-one;
5-(((R)-1-((R)-2-hydroxy-3-oxo-3-(4-(5-(trifluoromethyl)pyrimidin-2-yl)piperazin-1-yl)propoxy)-3-(trifluoromethoxy)propan-2-yl)amino)-4-(trifluoromethyl)pyridazin-3(2H)-one;
5-(((S)-1-((R)-2-hydroxy-3-oxo-3-(4-(5-(trifluoromethyl)pyrimidin-2-yl)piperazin-1-yl)propoxy)-3-(trifluoromethoxy)propan-2-yl)amino)-4-(trifluoromethyl)pyridazin-3(2H)-one;
5-((S)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;
5-((S)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;
5-((R)-1-(((R)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;
5-((R)-1-(((S)-2-hydroxy-3-oxo-3-(4-(5-(trifluoromethyl)pyrimidin-2-yl)piperazin-1-yl)propoxy)methyl)isoindolin-2-yl)-4-(trifluoromethyl)pyridazin-3(2H)-one;
5-((2S)-2-((2-hydroxy-3-oxo-3-(4-(5-(trifluoromethyl)pyrimidin-2-yl)piperazin-1-yl)propoxy)methyl)azetidin-1-yl)-4-(trifluoromethyl)pyridazin-3(2H)-one; and
5-(((S)-1-((R)-2-hydroxy-3-oxo-3-(4-(5-(trifluoromethyl)pyrimidin-2-yl)piperazin-1-yl)propoxy)-3-methoxypropan-2-yl)amino)-4-(trifluoromethyl)pyridazin-3(2H)-one;
or a pharmaceutically acceptable salt of any of the aforementioned.

37. A pharmaceutical composition comprising a compound of any one of claims 1-36, or a pharmaceutically acceptable salt thereof, and at least one pharmaceutically acceptable carrier.

38. A method of inhibiting the activity of PARP7 comprising contacting a compound of any one of claims 1-36, or a pharmaceutically acceptable salt thereof, with said PARP7.

39. A method of treating cancer in a patient in need of treatment comprising administering to said patient a therapeutically effective amount of a compound of any one of claims 1-36, or a pharmaceutically acceptable salt thereof.

40. The method of claim 39, wherein said cancer is breast cancer, cancer of the central nervous system, endometrium cancer, kidney cancer, large intestine cancer, lung cancer, oesophagus cancer, ovarian cancer, pancreatic cancer, prostate cancer, stomach cancer, head and neck cancer (upper aerodigestive cancer), urinary tract cancer, or colon cancer.
